(19) United States
(12) Patent Application Publication
(10) Pub. No.: US 2014/0044709 A1
(43) Pub. Date: Feb. 13, 2014

(54) TREATMENT OF HER2-POSITIVE CANCER WITH PACLITAXEL AND TRASTUZUMAB-MCC-DM1

(71) Applicants: Barbara Klencke, San Francisco, CA (US); Scott Holden, South San Francisco, CA (US); Alice Guardino, South San Francisco, CA (US); Betsy Althaus, South San Francisco, CA (US)

(72) Inventors: Barbara Klencke, San Francisco, CA (US); Scott Holden, South San Francisco, CA (US); Alice Guardino, South San Francisco, CA (US); Betsy Althaus, South San Francisco, CA (US)

(73) Assignee: GENENTECH, INC., South San Francisco, CA (US)

(21) Appl. No.: 13/991,580

(22) PCT Filed: Dec. 7, 2011
(86) PCT No.: PCT/US2011/063764

§ 371 Date: Oct. 21, 2013

Related U.S. Application Data
(60) Provisional application No. 61/421,385, filed on Dec. 9, 2010.

(51) Int. Cl. A61K 47/48; A61K 39/395; A61K 31/337

(52) U.S. Cl. CPC A61K 47/48723; A61K 31/337; A61K 39/39558
USPC 424/133.1; 424/183.1

(57) ABSTRACT

The present invention relates to combination therapy with paclitaxel, trastuzumab-MCC-DM1 and optionally, pertuzumab.

### RELATED APPLICATIONS

This application claims the benefit under 35 USC 119(e) of U.S. Provisional Application No. 61/421,385, filed Dec. 9, 2010, the contents of which are incorporated herein by reference.

### FIELD OF THE INVENTION

The present invention relates to methods of treating HER2-positive cancer, including HER2-positive metastatic breast cancer (MBC), with trastuzumab-MCC-DM1 (T-DM1) in combination with paclitaxel, and optionally in further combination with pertuzumab. Efficacious dosing regimens with low doses of T-DM1 and/or paclitaxel are further provided herein.

### BACKGROUND

### HER2-Positive Metatstatic Breast Cancer

Breast cancer is the most commonly diagnosed cancer among women, and is the second leading cause of cancer death after lung cancer. Approximately 180,000 women are diagnosed with breast cancer in the United States annually, of whom 40,000 will die of the disease (Jemal et al. 2008). Metastatic breast cancer (MBC) remains a therapeutic challenge and accounts for the vast majority of breast cancer-related mortality. The use of modern molecular techniques has demonstrated that breast cancer is a heterogeneous disease with a widely variable clinical course and response to therapy.

Protein overexpression or gene amplification of HER2 (also known as erbB2, neu, and p185HER2) is observed in approximately 20% of human breast cancers. Several lines of scientific and clinical evidence support a direct role for HER2 overexpression in the aggressive growth and poor clinical outcomes associated with these tumors (Slamon et al. 1987). Trastuzumab, a humanized monoclonal antibody directed against the extracellular region of HER2, is a highly effective treatment for both early-stage and metastatic HER2-positive breast cancer and has become the standard of care for these patients (Cobleigh et al. 2002; Slamon et al. 2001; Vogel et al. 2002; Marty et al. 2005; Piccart-Gebhart et al. 2005; Romond et al. 2005).

### T-DM1

Antibody-drug conjugates (ADCs) are monoclonal antibodies to which highly potent cytotoxic agents have been conjugated. They represent a novel approach to conferring selectivity to systemically administered anti-tumor therapeutics. ADCs are designed to focus the delivery of highly potent cytotoxic agents to tumor cells by targeting surface antigens that are tumor-specific and/or overexpressed. This approach potentially creates a more favorable therapeutic window for such agents than can be achieved by their administration as free drugs.

Trastuzumab-MCC-DM1 (T-DM1) is a novel ADC that has been studied as a single agent in patients with HER2-positive breast cancer. It is composed of the cytotoxic agent, DM1 (a thiol-containing maytansinoid anti-microtubule agent) conjugated to trastuzumab via SMCC, a linker molecule. The average drug-to-antibody ratio is approximately 3.5:1. T-DM1 binds to HER2 with an affinity similar to that of trastuzumab; such binding is required for its anti-tumor activity. It is hypothesized that after binding to HER2, T-DM1 undergoes receptor-mediated internalization, followed by intracellular release of DM1 and subsequent cytotoxicity.

A Phase I, open-label, dose-escalation study evaluating the safety and efficacy of T-DM1 as a single agent in patients with HER2-positive MBC whose disease progressed on a trastuzumab-containing chemotherapy regimen was conducted. The study was activated in April 2006, enrollment was completed in May 2008 with 52 patients dosed with T-DM1, and follow-up was completed in June 2009. Twenty-four patients were enrolled on the every-3-week dosing schedule, and 28 patients were enrolled on the weekly schedule. Prior to enrollment, patients had received nearly 2 years of trastuzumab (a median of 92 weeks for patients enrolled on the every-3-week schedule and 122 weeks for patients enrolled on the weekly schedule). The median number of prior chemotherapy agents for metastatic disease received by patients enrolled in this study was four for patients treated every 3 weeks and five for patients treated weekly.

Available data from the Phase I study are summarized below for all patients on both schedules. Treatment with T-DM1 was well tolerated. Toxicity has generally been mild, reversible, and noncumulative. No drug-related cardiac toxicity has been noted on either schedule, and no deaths have been reported other than those due to progressive disease (PD). The most common (i.e., >25%) Grade ≧2 adverse events reported for patients on either schedule were thrombocytopenia, nausea (weekly schedule), fatigue, and headaches. The only Grade 4 events reported to date have been transient and reversible thrombocytopenia occurring in one patient at the 2.4 mg/kg weekly dosing schedule and in two patients at the 4.8 mg/kg every-3-week dosing schedule. No clinically significant bleeding events have been observed. One serious adverse event (pulmonary hypertension) was considered possibly related to study drug. All other reported serious adverse events were not considered to be drug related.

Two of the 3 patients treated at the 4.8 mg/kg every-3-week dose level developed transient Grade 4 thrombocytopenia and Grade 2 elevations in liver function tests. The 3.6 mg/kg every-3-week dose level was expanded to 15 patients, and no DLTs were observed. No Grade 4 toxicity has been reported in these 15 patients. Therefore, the dose recommended for the Phase II study was 3.6 mg/kg every 3 weeks. The maximum tolerated dose (MTD) of 2.4 mg/kg for the weekly schedule was established when 2 of the 3 patients treated at the 2.9 mg/kg weekly dose level were unable to receive Day 8 dosing because of Grade 3 thrombocytopenia.

Five of the 24 patients treated on the every-3-week schedule, achieved a response (objective response rate [ORR]=21%). All responses were at doses at or below the MTD (3.6 mg/kg T-DM1 given every 3 weeks): one at 2.4 mg/kg T-DM1 (n=1) and five at 3.6 mg/kg T-DM1 (n=15, of which 9 had measurable disease) given every 3 weeks. Of the six responses, five have been confirmed according to the Response Evaluation Criteria in Solid Tumors (RECIST). There were 15 patients treated at the 3.6 mg/kg T-DM1 MTD level. Of these, 5 achieved a partial response (PR, 33%), as described above, and 7 had stable disease (SD). Two of the patients with SD have had durations of 505+ and 801+days.

Fifteen of the 28 patients treated on the weekly schedule achieved a response (ORR=54%). All responses were at doses at or below the MTD (2.4 mg/kg T-DM1 given weekly): three at 1.2 mg/kg T-DM1 (n=3), two at 1.6 mg/kg T-DM1 (n=3), two at 2.0 mg/kg T-DM 1 (n=3), and eight at 2.4 mg/kg T-DM1 (n=16) given weekly. Of the 15 responses, 13 have been confirmed according to RECIST.

Any apparent difference in ORR in patients treated with the weekly schedule compared to the every 3-week schedule disappears when considering only those patients with measurable disease and confirmed HER2 status by central review ( 7/15 [47%] vs. 5/11 [46%], weekly vs. every 3-week, respectively). The 6-month clinical benefit rate, defined as complete response (CR)+PR+SD>6 months, in patients treated with the weekly schedule is 18/28 (64%), and with every 3-week schedule is 13/24 (54%).

A Phase II, open-label study evaluating the safety and efficacy of T-DM1 in patients with HER2-positive MBC whose disease had progressed on a trastuzumab-containing chemotherapy regimen was conducted. In this study, T-DM1 was administered at a dose of 3.6 mg/kg every 3 weeks until PD or unacceptable toxicity. The study was activated in July 2007, and enrollment was completed in July 2008, with 112 patients enrolled (the planned enrollment was approximately 110 patients to ensure 100 evaluable patients). Patients with confirmed HER2-positive MBC whose disease had progressed on multiple lines of therapy, including HER2-directed therapy, were eligible.

The most common adverse events were fatigue, nausea, headache, constipation, and epistaxis. Grade 3-4 adverse events occurred in 40 patients; the most common adverse events were thrombocytopenia (n=8) and hypokalemia (n=6). In addition, 1 patient died from respiratory failure within 2 weeks of stopping T-DM1 therapy. The cause of death was considered to be PD by the investigator.

As of the data cutoff date, 107 patients were evaluable for efficacy. The median follow-up of this cohort was 4.4 months (19 weeks). There were 42 (39.3%) objective responses (CRs and PRs) in this group. Of these responses, 29 (27.1%) were confirmed by follow-up imaging. Of note, only 19 of the 107 patients had one post-baseline tumor assessment because of the limited follow-up. In addition, the response rate in the subgroup of patients with at least 6 months of follow-up or who had discontinued from the study (n=76) was 43.4% (n=33), with 38.2% (n=29) confirmed by follow-up imaging.

### Paclitaxel

Paclitaxel is a diterpenoid taxane derivative found in the bark and needles of T. brevifolia and is among the most active agents in the treatment of breast cancer. Paclitaxel binds to dimeric tubulin, preventing microtubule disassembly and ultimately leading to cell death. The role of paclitaxel in the treatment of breast cancer has been well established in both the adjuvant and metastatic settings.

The response rates for paclitaxel administered as a single agent to patients with MBC are 29-63% in first-line treatment (Wilson et al. 1994; Nabholtz et al. 1996; Seidman et al. 1995) and 21-56% in second-line treatment (Holmes et al. 1991; Abrams et al. 1995; Seidman et al. 1995; Fountzilas et al. 1996).

Two Phase III clinical trials of paclitaxel administered in combination with trastuzumab in patients with MBC have demonstrated improvements in response rate, time to progression (TTP), and overall survival in patients with HER2-positive MBC who were previously untreated for metastatic disease compared with those treated with paclitaxel alone.

### Pertuzumab

Pertuzumab is a humanized monoclonal antibody that is based on the human IgG1 (K) framework sequences. It consists of two heavy chains and two light chains. Like trastuzumab, pertuzumab is directed against the extracellular domain of HER2. However, it differs from trastuzumab in the epitope-binding regions of the light chain and heavy chain. As a result, pertuzumab binds to an epitope within what is known as a subdomain 2 of HER2, while the epitope from trastuzumab is localized to subdomain 4 (Cho et al. 2003; Franklin et al. 2004).

Pertuzumab acts by blocking the association of HER2 with other HER family members, including HER1 (epidermal growth factor receptor; EGFR), HER3, and HER4. This association is required for signaling in the presence of ligand via MAP-kinase and P13-kinase. As a result, pertuzumab inhibits ligand-initiated intracellular signaling. Inhibition of these signaling pathways can result in growth arrest and apoptosis, respectively (Hanahan and Weinberg 2000).

Because pertuzumab and trastuzumab bind at distinct epitopes on the HER2 receptor, ligand-activated downstream signaling is blocked by pertuzumab but not by trastuzumab. Pertuzumab, therefore, may not require HER2 overexpression to exert its activity as an anti-tumor agent. In addition, because of their complementary modes of action, the combination of pertuzumab and trastuzumab may have a potential role in HER2-overexpressing diseases.

The combination of pertuzumab with T-DM1 also appears to be synergistic in the HER2-positive KPL4 breast cancer xenograft model. A single dose of 3 mg/kg T-DM1, combined with weekly doses of 30 mg/kg (loading dose) and two maintenance doses of 15 mg/kg pertuzumab, led to substantial and prolonged tumor shrinkage in all 8 animals tested.

Pertuzumab has been evaluated as a single agent in five Phase II studies conducted in various cancer types, including MBC expressing low levels of HER2, non-small-cell lung cancer, hormone-refractory prostate cancer, and ovarian cancer. In general, pertuzumab was well tolerated.

Experience with pertuzumab in breast cancer includes a Phase II trial that evaluated pertuzumab as a single agent in the second- or third-line treatment of patients with MBC who had normal HER2 expression (Cortes et al. 2005) and in two Phase II studies of pertuzumab in combination with trastuzumab. The second Phase II study of full doses of pertuzumab and trastuzumab enrolled patients with previously-treated HER2-positive MBC who had previously received trastuzumab for MBC (Baselga et al. 2008; Gelmon et al. 2008) and demonstrated an acceptable safety profile, as well as evidence of clinical activity. In the 66 efficacy-evaluable patients, the objective response rate based on investigator assessment was 24% (11 PRs, 5 CRs), and 17 patients (25.8%) had SD for more than 6 months. The median duration of response in these 6 responders was 29 weeks. Clinical benefit rate was 50%. The most common adverse events were diarrhea, fatigue, nausea, and rash, with the majority of these being Grade 1-2. Four treatment-related Grade 3 adverse events were reported in 3 patients: 2 patients had diarrhea (resolved with supportive care) and 1 patient had 1) a rash and 2) a central line infection following the injection of contrast dye but prior to the administration of pertuzumab. Three of the 66 patients (4.5%) in this study had an LVEF decrease of ≧10% with an absolute LVEF value of <50%. One of the LVEF decreases was determined solely by local assessment, and not confirmed by central assessment (the patient was withdrawn due to PD). LVEF declines in the other 2 patients recovered, and the patients continued on protocol therapy. A Phase III trial has also been initiated in 200, which is a randomized study of trastuzumab and docetaxel with either pertuzumab or placebo for the first-line treatment of HER2-positive MBC.

### Outlook

Despite the advances that have been made, MBC is incurable. The primary goals of treatment remain to extend life and palliate symptoms while preserving quality of life. At present, no single regimen can be considered the global standard of care for advanced breast cancer. For patients with HER2-positive MBC, the combination of trastuzumab and chemotherapy is established as a standard treatment option based on positive results of two large pivotal trials. However, virtually all patients with HER2-positive MBC will eventually progress on available therapies. Opportunities remain to improve outcomes for patients with MBC.

The invention described herein meets the above-described needs and provides other benefits.

### SUMMARY

In one aspect, a method of treating HER2-positive cancer is provided, the method comprising administering a combination of paclitaxel and T-DM1 to a human patient having HER2-positive cancer, wherein paclitaxel is administered at a dosage of 65 mg/m² or 80 mg/m² weekly and T-DM1 is administered at a dosage ranging from 2.4-3.6 mg/kg every three weeks. In one embodiment, T-DM1 is administered at a dosage selected from 2.4, 3.0 and 3.6 mg/kg every three weeks. In a further embodiment, paclitaxel is administered at a dosage of 80 mg/m² weekly and T-DM1 is administered at a dosage selected from 2.4, 3.0 and 3.6 mg/kg every three weeks. In a further embodiment, paclitaxel is administered at a dosage of 65 mg/m² weekly and T-DM1 is administered at a dosage selected from 2.4, 3.0 and 3.6 mg/kg every three weeks. In a further embodiment, paclitaxel is administered at a dosage of 80 mg/m² weekly, T-DM1 is administered at a dosage selected from 2.4, 3.0 and 3.6 mg/kg every three weeks, and 420 mg pertuzumab is administered every three weeks. In one such embodiment, an initial loading dose of 840 mg of pertuzumab is administered, followed by the administering of 420 mg pertuzumab every three weeks. In a further embodiment, paclitaxel is administered at a dosage of 65 mg/m² weekly, T-DM1 is administered at a dosage selected from 2.4, 3.0 and 3.6 mg/kg every three weeks, and 420 mg pertuzumab is administered every three weeks. In one such embodiment, an initial loading dose of 840 mg of pertuzumab is administered, followed by the administering of 420 mg pertuzumab every three weeks.

In any of the above embodiments, the HER2-positive cancer to be treated is HER2-positive breast cancer. In one such embodiment, the HER2-positive breast cancer is metastatic. In any of the above embodiments, the HER2-positive cancer expresses HER2 at a 3+ level.

In a further aspect, a method of treating HER2-positive cancer is provided, the method comprising administering a combination of paclitaxel and T-DM1 to a human patient having HER2-positive cancer, wherein paclitaxel is administered at a dosage of 65 mg/m² or 80 mg/m² weekly and T-DM1 is administered at a dosage between 1.2-2.4 mg/kg weekly. In one embodiment, paclitaxel is administered at a dosage of 65 mg/m² or 80 mg/m² weekly and T-DM1 is administered at a dosage of 1.2, 1.6, 2.0 or 2.4 mg/kg weekly. In a further embodiment, paclitaxel is administered at a dosage of 65 mg/m² weekly and T-DM1 is administered at a dosage of 2.4 mg/kg weekly. In one such embodiment, 420 mg pertuzumab is administered every three weeks. In one such embodiment, an initial loading dose of 840 mg of pertuzumab is administered, followed by the administering of 420 mg pertuzumab every three weeks.

In any of the above embodiments, the HER2-positive cancer to be treated is HER2-positive breast cancer. In one such embodiment, the HER2-positive breast cancer is metastatic. In any of the above embodiments, the HER2-positive cancer expresses HER2 at a 3+ level. In any of the above embodiments, paclitaxel and T-DM1 are coformulated.

In a further aspect, a method of treating HER2-positive cancer is provided, the method comprising administering a combination of paclitaxel and T-DM1 to a human patient having HER2-positive cancer, wherein paclitaxel is administered at a dosage of 65 mg/m² or 80 mg/m² weekly and T-DM1 is administered at a dosage of 2.0 mg/kg every three weeks. In one embodiment, paclitaxel is administered at a dosage of 80 mg/m² weekly and T-DM1 is administered at a dosage of 2.0 mg/kg every three weeks. In another embodiment, paclitaxel is administered at a dosage of 65 mg/m² weekly and T-DM1 is administered at a dosage of 2.0 mg/kg every three weeks. In one such embodiment, the method further comprises administering 420 mg pertuzumab every three weeks. In one such embodiment, an initial loading dose of 840 mg of pertuzumab is administered, followed by the administering of 420 mg pertuzumab every three weeks. In any of the above embodiments, the HER2-positive cancer is HER2-positive breast cancer. In one such embodiment, the HER2-positive breast cancer is metastatic. In any of the above embodiments, the HER2-positive cancer expresses HER2 at a 3+ level.

In a further aspect, a method of treating HER2-positive cancer is provided, the method comprising administering a combination of paclitaxel and T-DM1 to a human patient having HER2-positive cancer, wherein paclitaxel is administered at a dosage of 65 mg/m² or 80 mg/m² weekly and T-DM1 is administered at a dosage between 1.2-2.0 mg/kg weekly. In one embodiment, paclitaxel is administered at a dosage of 65 mg/m² or 80 mg/m² weekly and T-DM1 is administered at a dosage of 1.2, 1.6 or 2.0 mg/kg weekly. In a further embodiment, paclitaxel is administered at a dosage of 65 mg/m² weekly and T-DM1 is administered at a dosage of 1.2 mg/kg weekly. In one such embodiment, the method comprises further comprising administering 420 mg pertuzumab every three weeks. In one such embodiment, an initial loading dose of 840 mg of pertuzumab is administered, followed by the administering of 420 mg pertuzumab every three weeks. In any of the above embodiments, the HER2-positive cancer is HER2-positive breast cancer. In one such embodiment, the HER2-positive breast cancer is metastatic. In any of the above embodiments, the HER2-positive cancer expresses HER2 at a 3+ level. In any of the above embodiments, paclitaxel and T-DM1 are coformulated.

### BRIEF DESCRIPTION OF THE FIGURES

FIG. 1 shows a dose escalation scheme for administration of a combination of paclitaxel and T-DM1.

FIG. 2 shows an alternative dose escalation scheme.

FIG. 3 shows addition of pertuzumab to a regimen of paclitaxel and T-DM1.

FIG. 4 shows a dose escalation scheme for administration of a combination of weekly paclitaxel and T-DM1.

FIG. 5 shows addition of pertuzumab to a regimen of weekly paclitaxel and T-DM1.

FIG. 6 shows study results.

### DETAILED DESCRIPTION OF EMBODIMENTS

### I. Definitions

“Trastuzumab-MCC-DM1” or “T-DM1,” is an antibody-drug conjugate (CAS Reg. No. 139504-50-0), which has the following structure:

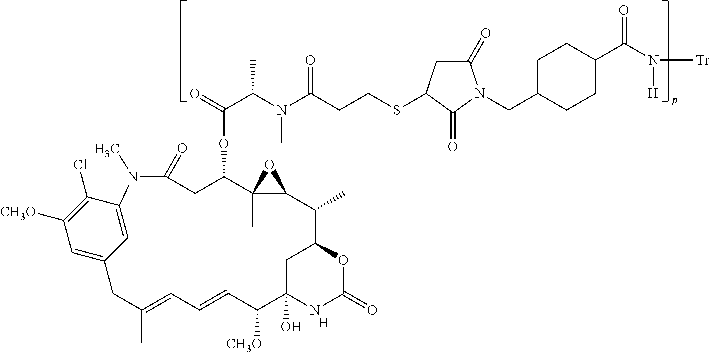

where Tr is trastuzumab linked through linker moiety MCC to the maytansinoid drug moiety DM1 (U.S. Pat. No. 5,208,020; U.S. Pat. No. 6,441,163). The drug to antibody ratio or drug loading is represented by p in the above structure of trastuzumab-MCC-DM1, and ranges in integer values from 1 to about 8. Trastuzumab-MCC-DM1 includes all mixtures of variously loaded and attached antibody-drug conjugates where 1, 2, 3, 4, 5, 6, 7, and 8 drug moieties are covalently attached to the antibody trastuzumab (U.S. Pat. No. 7,097,840; US 2005/0276812; US 2005/0166993).

“Paclitaxel” (TAXOL®, Bristol-Myers Squibb Oncology, Princeton N.J., CAS Reg. No. 33069-62-4) is a compound named as β-(benzoylamino)-α-hydroxy-,6,12b-bis (acetyloxy)-12-(benzoyloxy)-2a,3,4,4a,5,6,9,10,11,12,12a,12b-dodecahydro-4,11-dihydroxy-4a,8,13,13-tetramethyl-5-oxo-7,11-methano-1H-cyclodeca(3,4)benz(1,2-b) oxet-9-ylester,(2aR-(2a-α,4-β,4a-β,6-β,9-α (α-R*,β-S*),11-α,12-α,12a-α,2b-α))-benzenepropanoic acid, with the following structure;

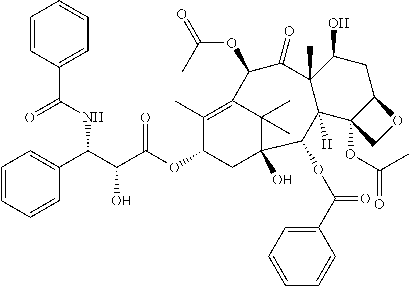 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

“Pertuzumab” (OMNITARG®, Genentech, Inc., South San Francisco Calif., CAS Reg. No. 380610-27-5) is a humanized monoclonal antibody that binds to extracellular domain II of HER2 and blocks its ability to dimerize with other HER receptors, as described in US 2005-0208043 A1.

The term “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “HER2-positive cancer” refers to a cancer comprising cells which have HER2 protein present at their cell surface. HER2 protein may be overexpressed, e.g., by gene amplification. Tumors overexpressing HER2 may be rated by immunohistochemical scores according to the number of copies of HER2 molecules expressed per cell, and can been determined biochemically: 0=0-10,000 copies/cell, 1+=at least about 200,000 copies/cell, 2+=at least about 500,000 copies/cell, 3+=at least about 2,000,000 copies/cell. Overexpression of HER2 at the 3+ level, which leads to ligand-independent activation of the tyrosine kinase (Hudziak et al., Proc. Natl. Acad. Sci. USA 84: 7159-7163 [1987]), occurs in approximately 30% of breast cancers, and in these patients, relapse-free survival and overall survival are diminished (Slamon et al., Science 244: 707-712 [1989]; Slamon et al., Science 235: 177-182 [1987]).

The terms “cancer” and “cancerous” refer to or describe the physiological condition in mammals that is typically characterized by unregulated cell growth/proliferation. Examples of cancer include, but are not limited to, carcinoma, lymphoma (e.g., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer.

### Abbreviations

AC cyclophosphamide. doxorubicin
ADC on mug conjugate
ADCC antibody dependent cell-mediated cytotoxicity
AE adverse event
ALP alkaline phosphatase
ATA anti-therapeutic antibodies
AUC area under the concentration-time curve
CNS central nervous system
Cₘₐₓ maximum serum concentration
Cₘᵢₙ minimum concentration
CHF congestive heart failure
CI confidence interval
CISH chromogenic in situ hybridization
CR complete response
CRO clinical research organization
CT computed tomography
DCP Dose Cohorts with Pertuzumab (Cohorts 5 and 9)
DFCI Dana Farber Cancer Institute
DLT dose-limiting toxicity
DM1 a derivative of maytansine
EC ethics committee
ECHO echocardiogram
ECOG Eastern Cooperative Oncology Group
ECD extracellular domain
EDC Electronic Data Capture
EGFR epidermal growth factor receptor
FDA Food and Drug Administration
FISH fluorescence in situ hybridization
GCP Good Clinical Practice
GFR glomerular filtration rate
ICH International Conference on Harmonisation
ICF Informed Consent Form
IHC immunohistochemistry
IMP investigational medicinal product
IND Investigational New Drug
IRB institutional review board
IUD intrauterine device
IUS intrauterine system
IV intravenous
IVRS interactive voice response system
LVEF left ventricular ejection fraction
LVSD left ventricular systolic dysfunction
MBC metastatic breast cancer
MRI magnetic resonance imaging
MTD maximum tolerated dose
MUGA multigated acquisition (scan)
NCI CTCAE National Cancer Institute Common Terminology Criteria for Adverse Events
NSCLC non small cell lung cancer
NYHA New York Heart Association
ORR objective response rate
PD progressive disease
PFS progression-free survival
PK pharmacokinetic
PR partial response
RECIST Response Evaluation Criteria in Solid Tumors
SAE serious adverse event
SD stable disease
SWFI Sterile Water for Injection
T-DM1 trastuzumab-MCC-DM1
TTP time to progression
ULN upper limit of normal

### II. Embodiments of the Invention

The present invention is based, in part, on a Phase Ib, open label, dose-escalation study of the safety, tolerability, and pharmacokinetics of trastuzumab-MCC-DM1, paclitaxel, and pertuzumab administered intravenously to patients with HER2-positive, locally advanced or metastatic breast cancer who have previously received a trastuzumab-containing regimen.

### 1. Clinical Study Objectives

### 1.1 Primary Objectives

### 1.1.1 Primary Objectives for Parts 1 and 3: Dose-Escalation Phase of T-DM1 and Paclitaxel

The primary objectives for Parts 1 and 3 of this study are as follows:
To characterize the safety and tolerability of the combination of T-DM1 and paclitaxel in patients with HER2-positive, locally advanced or metastatic breast cancer who have previously received HER2-directed therapy
To determine the MTD and DLTs of the combination of T-DM1 and paclitaxel when T-DM1 is administered on either an every-3-week or weekly schedule
To evaluate the pharmacokinetics of T-DM1 in the presence of paclitaxel when T-DM1 is administered on either an every-3-week or weekly schedule
To evaluate the pharmacokinetics of paclitaxel in the presence and absence of T-DM1 when T-DM1 is administered on either an every-3-week or weekly schedule

### 1.1.2 Primary Objective for Parts 2 and 4: Addition of Pertuzumab to T-DM1 and Paclitaxel

The primary objective for Parts 2 and 4 of this study is as follows:
To characterize the safety and tolerability of the combination of pertuzumab added to the recommended dose of T-DM1 and paclitaxel, as determined in Parts 1 and 3 of the study, in patients with HER2-positive, locally advanced or metastatic breast cancer who have previously received HER2-directed therapy

### 1.2. Secondary Objectives

The secondary objectives for this study are as follows:
To make a preliminary assessment of the efficacy of the combination of T-DM1 and paclitaxel (and pertuzumab, if applicable), as measured by ORR based on investigator assessment using modified RECIST (v.1.0) (Therasse P, Arbuck S G, Eisenhauser E A, Wanders J, Kaplan R S, Rubinstein L, et al. New guidelines to evaluate the response to treatment in solid tumors. J Natl Cancer Inst 2000; 92:205-16)
To make a preliminary assessment of the efficacy of the combination of T-DM1 and paclitaxel (and pertuzumab, if applicable), as measured by PFS, duration of response, and clinical benefit rate
   Clinical benefit rate is defined as clinical response or SD of >_{—}6 months duration as assessed by the investigator.
To assess the development of anti-therapeutic antibodies to T-DM1 when T-DM1 is administered in combination with paclitaxel

### 1.3. Exploratory Objectives

The exploratory objectives for this study are as follows:
To assess the relationship of HER2 status (determined by central laboratory testing) with ORR
To assess the safety of T-DM1 (and pertuzumab, if applicable) when administered to patients who have developed isolated brain metastases while receiving study treatment
To assess the etiology of T-DM1—associated thrombocytopenia by characterizing the structure and function of platelets collected from select patients treated with T-DM1

### 2. Clinical Study Design

### 2.1 Description of the Study

This is a Phase Ib, multi-institutional, open-label, dose-escalation study designed to evaluate the safety, tolerability, and pharmacokinetics of T-DM1 administered by intravenous (IV) infusion in combination with paclitaxel (and pertuzumab, if applicable) in patients with HER2-positive, locally advanced or metastatic breast cancer who have previously received HER2-directed therapy. The study will be conducted in four parts (Parts 1 and 3 for dose escalation and Parts 2 and 4 for the addition of pertuzumab) as described below. Parts 2 and 3 will start upon completion of Part 1, and Part 4 will start upon completion of Part 3.

### 2.1.1 Part 1: Dose Escalation of T-DM1 and Paclitaxel

In Part 1 of this study, patients will receive a combination of T-DM1 (given every 3 weeks or weekly) and paclitaxel (given weekly) in repeated cycles until progression of disease or unacceptable toxicity. Both schedules of T-DM1 (every-3-week and weekly) will be investigated. It is anticipated that approximately five dose cohorts of T-DM1 and paclitaxel will be evaluated, with starting doses of paclitaxel 65 mg/m² weekly and T-DM1 2.4 mg/kg every 3 weeks (see Table 1). During Cycle 1, paclitaxel dosing will occur on Day—1 (the day prior to the first dose of T-DM1 on Day 1) rather than on Day 1 as in subsequent cycles to allow for the determination of paclitaxel pharmacokinetics in the absence of T-DM1.

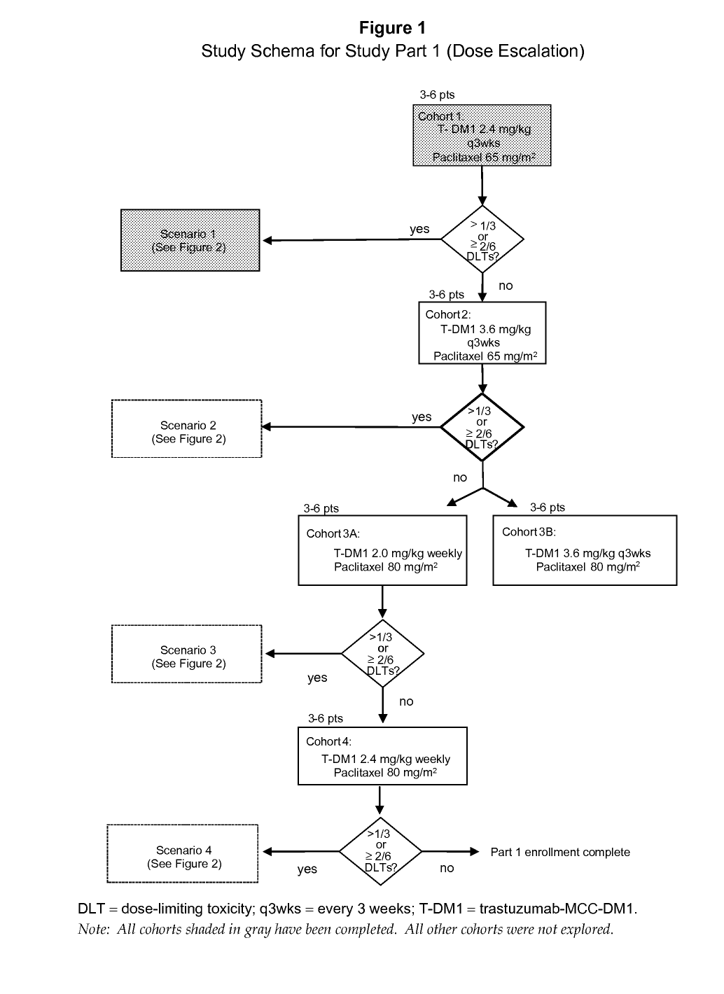

For dose escalation, a traditional 3+3 dose-escalation scheme will be used (see FIG. 1). A minimum of 3 patients will be enrolled into each dose cohort (see Table 1 and FIG. 1) and followed for a minimum of 23 days (the DLT observation period, defined as Cycle 1, Days—1 to 21, plus the assessments prior to drug administration on Cycle 2, Day 1) before enrollment in the next dose cohort begins. Any patient who does not complete the full 23-day DLT observation period for any reason other than a DLT will be considered nonevaluable for dose-escalation decisions and will be replaced by an additional patient in that same dose cohort.

If the last patient enrolled in a given dose cohort completes dosing through the DLT observation period without experiencing a DLT, another 3 patients can be enrolled at the next higher dose cohort (see Table 1). If 1 patient experiences a DLT, an additional 3 patients will be enrolled in the same dose cohort. If there are no further DLTs in the expanded cohort of 6 patients, dose escalation will continue.

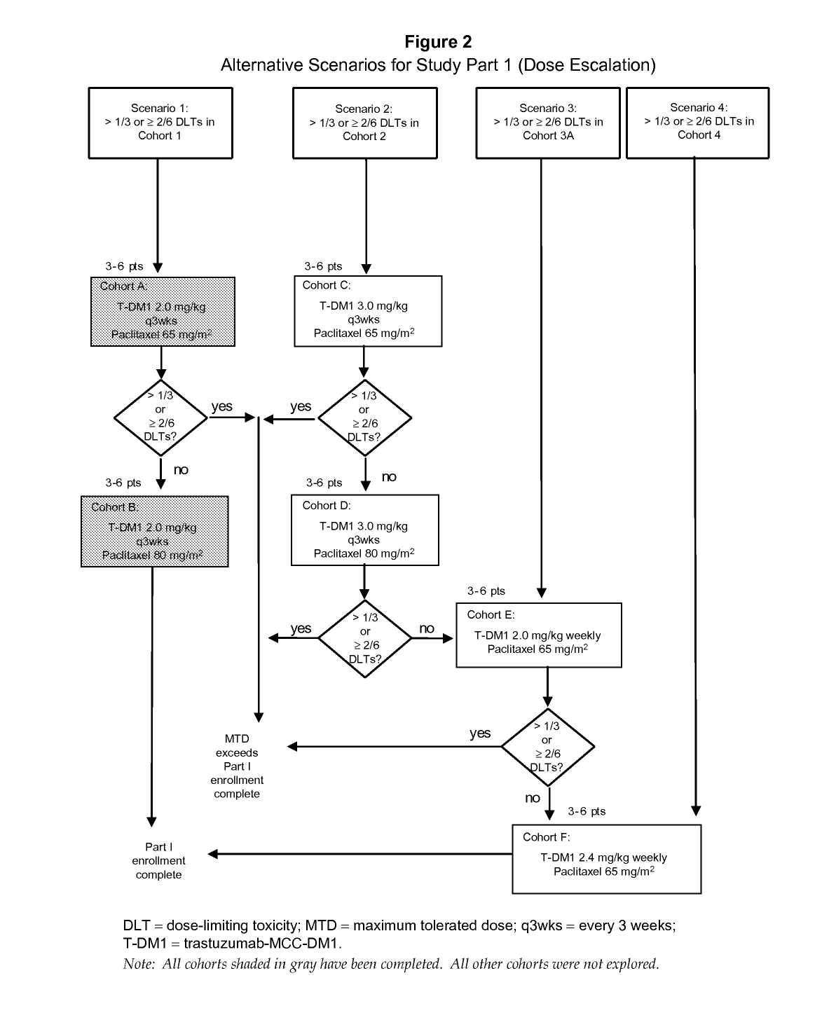

If 2 or more patients in the dose cohort experience a DLT, alternative dose cohorts may be opened, as described in FIG. 2 and Table 2. If more than 1 of 3 patients or 2 or more of 6 patients experience a DLT, and all of the appropriate alternative dose cohorts (i.e., those that follow the dose cohort in Table 1 at which DLT was observed) have been tested, no further dose escalation will occur. The MTD will be defined as the highest dose at which 0 of 3 patients or 1 of 6 patients experiences a DLT. Patients in Part 1 who permanently discontinue paclitaxel study treatment may be allowed to escalate the T-DM1 dose to 3.6 mg/kg every 3 weeks, per the clinical judgment of the investigator.

If the MTD of T-DM1 and paclitaxel has not been reached by Dose Cohort 2, the weekly (Dose Cohort 3A) and every-3-week (Dose Cohort 3B) schedules of T-DM1 will be tested concurrently. Assignment to these cohorts will be made on an alternating basis (i.e., the first patient will be enrolled in Dose Cohort 3A, the second in Dose Cohort 3B, and so on). Alternating enrollment into these simultaneous cohorts will continue until 3 patients have been enrolled in each cohort or until a DLT is observed, leading to expansion (if there is a DLT in 1 of 3 patients) or discontinuation of enrollment (if there is a DLT in 2 or more of 6 patients) into the Dose Cohort at which the DLT was observed; if DLTs are observed in one cohort and not the other, enrollment into the cohort in which no DLT has been observed may continue. In order to gain an understanding of the tolerability of T-DM1 in combination with the chronic administration of paclitaxel, an attempt should be made to treat patients with a minimum of 12 consecutive weeks of paclitaxel.

TABLE 1
Description of Dose Cohorts during Study Part 1
(Dose Escalation of T-DM1 and Paclitaxel)

| n | Dose Cohort | T-DM1a, b | Paclitaxel (weekly)^{b} |
|---|---|---|---|
| 3-6 | 1^{c} | 2.4 mg/kg every 3 weeks | 65 mg/m² |
| | (Starting Dose) | | |
| 3-6 | 2^{d} | 3.6 mg/kg every 3 weeks | 65 mg/m² |
| 3-6 | 3A^{ej} | 2.0 mg/kg weekly | 80 mg/m2^{e} |
| 3-6 | 3B | 3.6 mg/kg every 3 weeks | 80 mg/m² |
| 3-6 | 4^{g} | 2.4 mg/kg weekly | 80 mg/m2^{g} |

DLT = dose-limiting toxicity;
MTD = maximum tolerated dose;
T-DM1 = trastuzumab-MCC-DM1.
^{a}Cycles are 21 days for patients receiving T-DM1 on the every-3-week schedule (one dose per cycle) as well as for those on the weekly schedule (three doses per cycle).
^{b}During the DLT observation period, treatment delays because of toxicity should be reported as DLTs. In Cycles 2 and beyond, treatment delays of up to 42 days from last dose for the every-3-week regimen, and 28 days from the last dose for the weekly regimen are otherwise permitted to assess and allow for the resolution or treatment of complications.
^{c}If ≧1/3 or ≧2/6 patients experience DLTs in this dose cohort, additional cohort(s) may be opened according to Scenario 1 (see Table 2 and FIG. 2) at the discretion of the Medical Monitor.
^{d}If ≧1/3 or ≧2/6 patients experience DLTs in this dose cohort, additional cohort(s) may be opened according to Scenario 2 (see Table 2 and FIG. 2) at the discretion of the Medical Monitor.
^{e}Enrollment into Dose Cohorts 3A and 3B will occur simultaneously. If Dose Cohort 3A is tolerated, dose escalation will continue to Dose Cohort 4.
^{f}If ≧1/3 or ≧2/6 patients experience DLTs in this dose cohort, additional cohort(s) may be opened according to Scenario 3 (see Table 2 and FIG. 2) at the discretion of the Medical Monitor.
^{g}If ≧1/3 or ≧2/6 patients experience DLTs in this dose cohort, additional cohort(s) may be opened according to Scenario 4 (see Table 2 and FIG. 2) at the discretion of the Medical Monitor.

TABLE 2
Description of Alternative Dose Cohorts during Study Part 1
(Dose Escalation of T-DM1 and Paclitaxel)

| n | Dose Cohort | T-DM1^{a,b} | Paclitaxel (weekly)^{b} |
|---|---|---|---|
| 3-6 | A | 2.0 mg/kg every 3 weeks | 65 mg/m² |
| 3-6 | B | 2.0 mg/kg every 3 weeks | 80 mg/m² |
| 3-6 | C | 3.0 mg/kg every 3 weeks | 65 mg/m² |
| 3-6 | D | 3.0 mg/kg every 3 weeks | 80 mg/m² |
| 3-6 | E | 2.0 mg/kg weekly | 65 mg/m² |
| 3-6 | F | 2.4 mg/kg weekly | 65 mg/m² |

DLT = dose-limiting toxicity;
MTD = maximum tolerated dose;
T-DM1 = trastuzumab-MCC-DM1.
^{a}Cycles are 21 days for patients receiving T-DM1 on the every-3-week schedule (one dose per cycle) as well as for those on the weekly schedule (three doses per cycle).
^{b}During the DLT observation period, treatment delays because of toxicity should be reported as DLTs. In Cycles 2 and beyond, treatment delays of up to 42 days from last dose for the every-3-week regimen, and 28 days from the last dose for the weekly regimen are otherwise permitted to assess and allow for the resolution or treatment of complications.

### 2.1.2 Part 2: Addition of Pertuzumab to T-DM1 and Paclitaxel

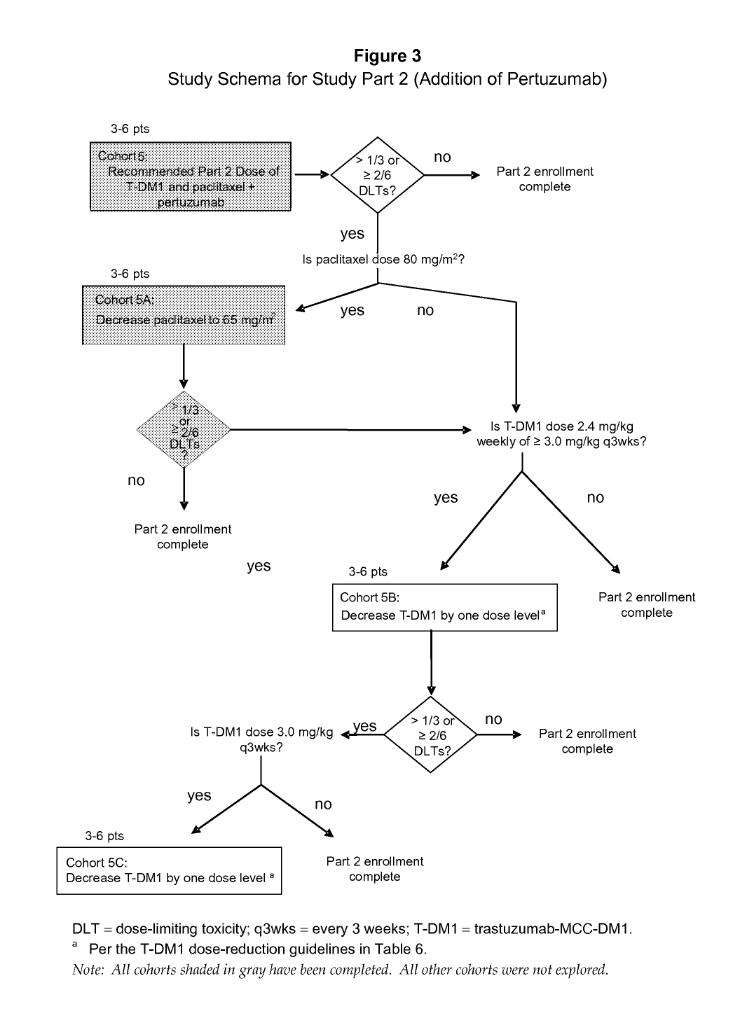

Once a safe and tolerable dose and schedule of T-DM1 administered in combination with paclitaxel is identified, the recommended Part 2 dose of this combination will be determined, and the safety and tolerability of this regimen combined with pertuzumab will be tested (see FIG. 3 and Table 3). For patients in this part of the study, the DLT observation period will be 22 days (defined as Cycle 1, Days 1 to 21, plus the assessments prior to drug administration on Cycle 2, Day 1). If pertuzumab is not tolerated in combination with the recommended Part 2 dose of T-DM1 and paclitaxel, the dose of paclitaxel will first be decreased to 65 mg/m², if applicable (see Table 3 and FIG. 3). If the recommended Part 2 dose of paclitaxel in combination with T-DM1 is already 65 mg/m², additional cohort(s) will be opened to receive a decreased dose of T-DM1 that is no less than 2.4 mg/kg every 3 weeks or 2.0 mg/kg weekly as described in Table 3. The dose-reduction algorithm for T-DM1 during Part 2 of the study will be the same as that described in Table 6.

Patients in Part 2 who permanently discontinue paclitaxel study treatment may be allowed to escalate the T-DM1 dose to 3.6 mg/kg every 3 weeks, per the clinical judgment of the investigator.

TABLE 3
Description of Dose Cohorts during Study Part 2 (Addition of Pertuzumab)

| n | Dose Cohort | T-DM1 and Paclitaxel^{a,b} | Pertuzumab |
|---|---|---|---|
| 3-6 | 5 | Recommended Part 2 dose of | Full dose (840 mg loading dose; |
| | | paclitaxel and weekly T-DM1 as | 420 mg in subsequent cycles) |
| | | determined in Part 1 | |
| 3-6 | 5A | Decrease paclitaxel to 65 mg/m2^{c} | Full dose (840 mg loading dose; |
| | | | 420 mg in subsequent cycles) |
| 3-6 | 5B | Decrease T-DM1 by one | Full dose (840 mg loading dose; |
| | | dose level^{d} | 420 mg in subsequent cycles) |
| 3-6 | 5C | Decrease T-DM1 by one | Full dose (840 mg loading dose; |
| | | additional dose level^{e} | 420 mg in subsequent cycles) |

MTD = maximum tolerated dose;
T-DM1 = trastuzumab-MCC-DM1.
^{a}Cycles are 21 days for patients receiving T-DM1 on the every-3-week schedule (one dose per cycle) as well as for those on the weekly schedule (three doses per cycle).
^{b}During the DLT observation period, treatment delays because of toxicity should be reported as DLTs. In Cycles 2 and beyond, treatment delays of up to 42 days from last dose for the every-3-week regimen, and 28 days from the last dose for the weekly regimen are otherwise permitted to assess and allow for the resolution or treatment of complications.
^{c}If Dose Cohort 5 is receiving paclitaxel 80 mg/m²; otherwise, proceed to Dose Cohort 5B if the T-DM1 dose is 2.4 mg/kg weekly or is ≧3.0 mg/kg every 3 weeks.
^{d}Decrease T-DM1 by one dose level (as described herein) to a dose of no less than 2.0 mg/kg weekly or 3.0 mg/kg every 3 weeks.
^{e}Decrease T-DM1 by one dose level (as described herein) to a dose of no less than 2.4 mg/kg every 3 weeks.

### 2.1.3 Part 3: Dose Escalation of Weekly T-DM1 and Paclitaxel

In Part 3 of this study, patients will receive a combination of weekly T-DM1 and weekly paclitaxel in repeated cycles until progression of disease or unacceptable toxicity. It is anticipated that approximately three dose cohorts of T-DM1 and paclitaxel will be evaluated, with starting doses of paclitaxel 65 mg/m² weekly and T-DM1 1.2 mg/kg weekly (see Table 4). During Cycle 1, paclitaxel dosing will occur on Day—1 (the day prior to the first dose of T-DM1 on Day 1) rather than on Day 1 as in subsequent cycles to allow for the determination of paclitaxel pharmacokinetics in the absence of T-DM1.

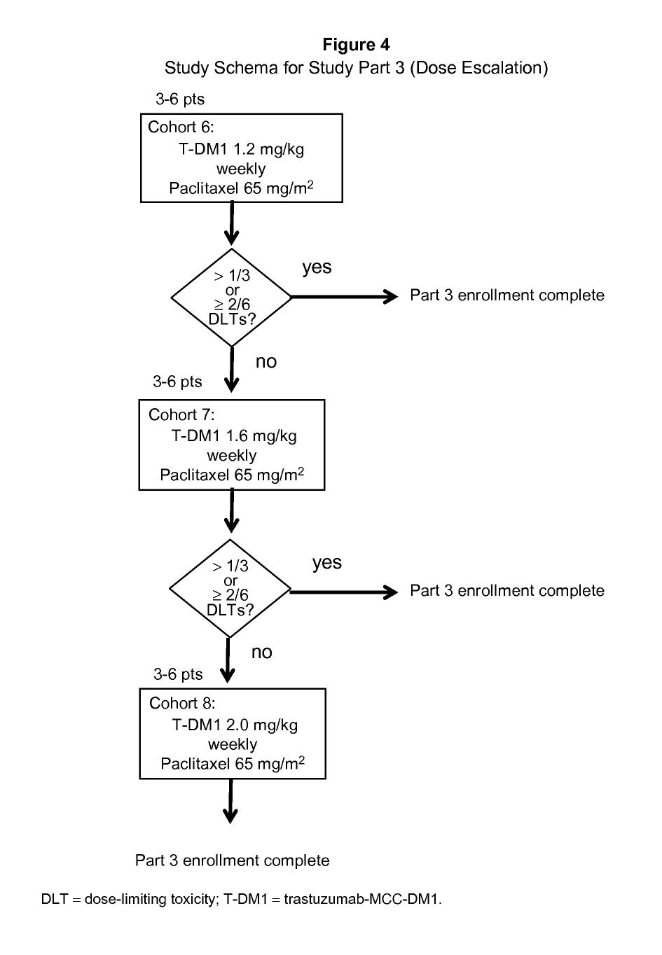

For dose escalation, a traditional 3+3 dose-escalation scheme will be used (see FIG. 4). A minimum of 3 patients will be enrolled in each dose cohort (see Table 4 and FIG. 4) and followed for a minimum of 23 days (the DLT observation period, defined as Cycle 1, Days 1 to 21, plus the assessments prior to drug administration on Cycle 2, Day 1) before enrollment in the next dose cohort begins. Any patient who does not complete the full 23-day DLT observation period for any reason other than a DLT will be considered nonevaluable for dose escalation decisions and will be replaced by an additional patient in that same dose cohort.

If the last patient enrolled in a given dose cohort completes dosing through the DLT observation period without experiencing a DLT, another 3 patients can be enrolled at the next higher dose cohort (see Table 4). If 1 patient experiences a DLT, an additional 3 patients will be enrolled in the same dose cohort. If there are no further DLTs in the expanded cohort of 6 patients, dose escalation will continue. If more than 1 of 3 patients or 2 or more of 6 patients experience a DLT, no further dose escalation will occur. The MTD will be defined as the highest dose at which 0 of 3 patients or 1 of 6 patients experiences a DLT. Patients in Part 3 who permanently discontinue paclitaxel study treatment will not be allowed to dose escalate the T-DM1 dose.

To gain an understanding of the tolerability of T-DM1 in combination with chronic administration of paclitaxel, an attempt should be made to treat patients with a minimum of 12 consecutive weeks of paclitaxel.

TABLE 4
Description of Dose Cohorts during Study Part 3
(Dose Escalation of Weekly T-DM1 and Paclitaxel)

| n | Dose Cohort | T-DM1a, b | Paclitaxel (weekly)^{b} |
|---|---|---|---|
| 3-6 | 6 | 1.2 mg/kg weekly | 65 mg/m² |
| 3-6 | 7 | 1.6 mg/kg weekly | 65 mg/m² |
| 3-6 | 8 | 2.0 mg/kg weekly | 65 mg/m² |

T-DM1 = trastuzumab-MCC-DM1.
^{a}Cycles are 21 days; patients receive T-DM1 on the weekly schedule (three doses per cycle).
^{b}During the DLT observation period, treatment delays due to toxicity should be reported as DLTs. In Cycles 2 and beyond, treatment delays of up to 28 days from last study treatment dose are otherwise permitted to assess and allow for the resolution or treatment of complications.

### 2.1.4 Part 4: Addition of Pertuzumab to Weekly T-DM1 and Paclitaxel

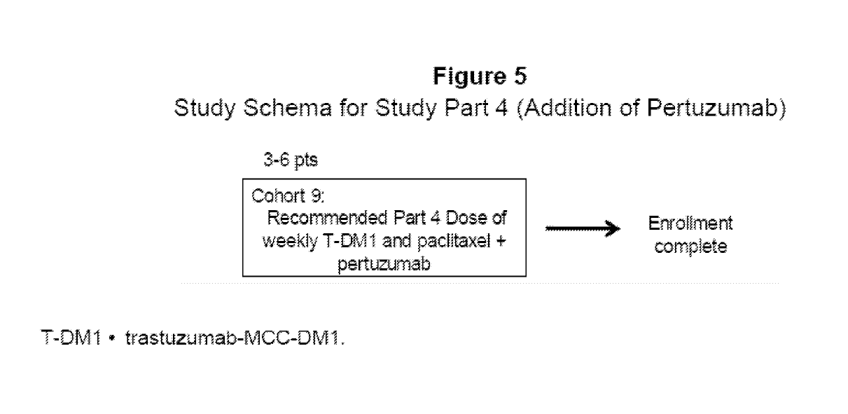

Once a safe and tolerable dose of weekly T-DM1 administered in combination with paclitaxel is identified in Part 3, the recommended Part 4 dose of this combination will be determined, and the safety and tolerability of this regimen combined with pertuzumab will be tested (see FIG. 5 and Table 5). For patients in Part 4 of the study, the DLT observation period will be 22 days (defined as Cycle 1, Days 1 to 21, plus the assessments prior to drug administration on Cycle 2, Day 1).

If pertuzumab is not tolerated in combination with the recommended Part-4 dose of T-DM1 and paclitaxel, additional cohort(s) may be added to test a decreased dose of T-DM1 that is no less than 1.2 mg/kg weekly. The dose-reduction algorithm for T-DM1 during Part 4 of the study will be the same as that described in Table 6. The decision about whether to open additional cohort(s) will be made on the basis of the risk/benefit data seen in Parts 3 and 4 at that time. Patients in Part 4 who permanently discontinue paclitaxel study treatment will not be allowed to dose escalate the T-DM1 dose.

TABLE 5
Description of Dose Cohort during
Study Part 4 (Addition of Pertuzumab)

| | Dose | | |
|---|---|---|---|
| N | Cohort | T-DM1 and Paclitaxel^{a,b} | Pertuzumab |
| 3-6 | 9 | Recommended Part 4 dose of | Full dose (840 mg loading |
| | | paclitaxel and weekly T-DM1 | dose; 420 mg in |
| | | as determined in Part 3 | subsequent cycles) |

T-DM1 = trastuzumab-MCC-DM1.
^{a}Cycles are 21 days; patients receive T-DM1 on the weekly schedule (three doses per cycle).
^{b}During the DLT observation period, treatment delays due to toxicity should be reported as DLTs. In Cycles 2 and beyond, treatment delays of up to 42 days from last dose for the every-3-week regimen and 28 days from the last dose for the weekly regimen are otherwise permitted to assess and allow for the resolution or treatment of complications.

### 2.1.5 Study Continuation

Enrollment into the study will stop when the recommended Part 2 and Part 4 doses of T-DM1 and paclitaxel have been determined, and when the addition of pertuzumab to these doses has been tolerated, or dose-reduction to minimum doses of T-DM1 and paclitaxel (as described in Tables 6 and 7) in combination with pertuzumab has occurred. Approximately 36 patients will be enrolled at up to four sites in the United States. The enrollment period will span approximately 14 months.

Patients without a DLT will be eligible to receive additional infusions of paclitaxel and T-DM1 (and pertuzumab, if applicable) at the same dose levels with a minimum interval of 7 days (+3 days) for paclitaxel and of either 7 days (+3 days; for the weekly schedule) or 17 days (21 days±3 days; for the every-3-week schedule) for T-DM1 (and 21 days [±3 days] for pertuzumab, if applicable). To be eligible to receive additional infusions of these drugs for up to 12 months from the time they enroll in the study, patients must not meet the criteria for PD and have acceptable toxicity and adequate cardiac function.

Patients who have demonstrated control of their systemic disease (defined for this purpose as objective response or SD maintained for at least 3 months) from T DM1-based therapy but who have developed isolated brain metastases that are treatable with radiation will be allowed to continue to receive therapy with T-DM1 and paclitaxel (and pertuzumab, if applicable) on study until they either experience systemic progression of their disease and/or further progression in the brain (i.e., recurrence at the site of prior therapy or emergence of a new lesion based on investigator assessment). Patients must not miss more than one cycle (42 days from last dose for the every-3-week regimen and 28 days from the last dose for the weekly regimen) for the treatment of their brain disease and must have an Eastern Cooperative Oncology Group (ECOG) performance status of 0, 1, or 2 to continue on therapy.

Patients may withdraw from the study at any time or may be withdrawn by the treating investigator if it is felt to be in the patient's best interest. If T-DM1 (and/or pertuzumab, if applicable) is not commercially available at the time a patient has completed 12 months of therapy, a treatment-extension protocol may be available for patients who continue to meet treatment criteria after having completed 12 months of therapy unless the study is terminated early. Patients who discontinue paclitaxel (and/or pertuzumab, if applicable) because of an adverse event may continue to receive T-DM1 on their previously determined schedule. Patients who discontinue T-DM1 because of an adverse event or other reason will discontinue all study treatment but will continue to be followed for 30 days for adverse events and serious adverse events and every 6 weeks for disease progression until the initiation of another anti-cancer therapy, patient- or investigator-initiated withdrawal from the study, or study termination by the Sponsor.

### 2.1.6 Patient Monitoring

All patients will be closely monitored for safety and tolerability during all cycles of therapy, at the treatment termination visit, and during the follow-up period. Patients will be clinically assessed before the infusion on Day 1 of each cycle (or Day 1 of Cycle 1 for patients in Parts 1 and 3 of the study) and additionally as needed. Radiographic assessment of disease status will be performed at screening, at the end of Cycle 2, and every two cycles thereafter throughout the duration of the study. Disease status (CR, PR, SD, or PD) will be assessed using modified RECIST (v.1.0). The definition of PD has been modified to be the following: At least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression.) Echocardiogram (ECHO) or multigated acquisition (MUGA) scans will be performed at screening, at the end of Cycle 2, and then every three cycles thereafter throughout the treatment period. Any patient with a significant decline in ejection fraction or with symptomatic CHF will be withdrawn from study treatment.

Pharmacokinetic (PK) sampling will be performed for all patients who receive paclitaxel and T-DM1 in Parts 1 and 3. Pharmacokinetic sampling will not be performed in patients who receive paclitaxel, T-DM1, and pertuzumab (i.e., patients in Dose Cohorts 5 and 9 [and Dose Cohorts 5A-5C, if applicable]) in order to avoid a potential confounding effect of the presence of pertuzumab on the measurement of T-DM1 levels. Serum concentrations of T-DM1 (conjugate), total trastuzumab (free trastuzumab+trastuzumab conjugated to DM1), and plasma concentrations of DM1 will be determined. In addition, plasma concentrations of paclitaxel will be determined. Relevant PK parameters for T-DM1 and paclitaxel (e.g., maximum concentration _{[Cmax]}, minimum trough concentration [Cmin], area under the concentration-time curve [AUC], volume of distribution, clearance, and elimination half-life) will be determined where possible using noncompartmental and/or population methods. Serum samples will also be analyzed for the presence of anti-therapeutic antibodies to T-DM1.

### 2.1.7 Definition of Dose-Limiting Toxicity

For Parts 1 and 3 of the study, the DLT observation period is Days—1 to 21 of Cycle 1, plus the assessments prior to drug administration on Cycle 2, Day 1 (23 days). For Parts 2 and 4 of the study (patients receiving pertuzumab), the DLT observation period includes Days 1 to 21 of Cycle 1, plus the assessments prior to drug administration on Cycle 2, Day 1 (22 days). In this study, a DLT will be defined as any of the following:
Grade ≧3 non-hematologic adverse event that is not due to disease progression or another clearly identifiable cause with the following exceptions: Alopecia of any grade, Grade 3 diarrhea that responds to standard-of-care therapy, Grade 3 nausea or vomiting in the absence of premedication that responds to standard-of-care therapy
Grade ≧3 elevation of serum bilirubin, hepatic transaminases (ALT or AST), or alkaline phosphatase (ALP) lasting >72 hours, with the following exception: For patients with Grade 2 hepatic transaminase levels at baseline (≦5× the upper limit of normal [ULN]) as a result of liver or bone metastases, a hepatic transaminase or ALP level≧10×ULN will be considered to be a DLT.
Grade ≧4 thrombocytopenia
Grade ≧4 neutropenia (absolute neutrophil count <500/cells/mm³) lasting >72 hours or accompanied by a fever (oral or tympanic temperature >101.3° F. or 38.5° C.)
Any subjectively intolerable toxicity felt by the investigator to be related to T-DM1, paclitaxel, or pertuzumab
Any treatment-related toxicity prompting a dose delay or modification of T-DM1 during the DLT observation period, such as prompting a dose reduction at Cycle 2, Day 1
Any treatment-related toxicity prompting more than one weekly paclitaxel dose delay or any weekly paclitaxel dose modification during the DLT observation period. One weekly paclitaxel dose delay during the DLT observation period will not constitute a DLT Toxicity will be graded according to the National Cancer Institute Common Terminology Criteria for Adverse Events (NCI CTCAE), Version 3.0.

### 2.2 Rationale for the Dose and Regimen

In a Phase I study, two dosing schedules of T-DM1 were tested—one in which T-DM1 was administered weekly, and one in which T-DM1 was administered every 3 weeks. The MTD of T-DM1 administered by IV infusion weekly was 2.4 mg/kg and the MTD of T-DM1 administered every 3 weeks was 3.6 mg/kg. DLTs consisted of Grade 3-4 thrombocytopenia that prevented retreatment in 2 of 3 patients treated at 2.9 mg/kg on the weekly schedule and in 2 of 3 patients treated at 4.8 mg/kg on the every-3-week schedule. Grade ≧2 adverse events at the MTD of either schedule were infrequent and manageable. The most common study drug-related adverse events seen in this study were fatigue, nausea, thrombocytopenia, transaminase elevations, headache, constipation, and anorexia.

Both the weekly and every-3-week T-DM1 schedules are well tolerated and associated with significant clinical activity as described previously. In addition, an ongoing Phase II study has shown similar tolerability of T-DM1 at a dose of 3.6 mg/kg administered every 3 weeks, with only a small percentage of patients (3 of 112 patients) requiring dose reduction. No cardiotoxicity has been observed to date in patients receiving either weekly or every-3-week T-DM1 in either of these studies.

Given the overlapping toxicities of T-DM1 and paclitaxel, and the unknown effect of paclitaxel on the pharmacokinetics of T-DM1, both the weekly and every-3-week T-DM1 regimens will be studied in combination with weekly paclitaxel in this study. Pertuzumab will be added once the recommended Part 2 and Part 4 doses for T-DM1 in combination with paclitaxel have been established.

The current Food and Drug Administration (FDA)-approved dose of paclitaxel for MBC is 175 mg/m² administered W every 3 weeks. However, several trials have explored dose-dense regimens of both paclitaxel and docetaxel in an attempt to increase therapeutic efficacy and reduce toxicity. Several Phase II trials have explored the combination trastuzumab in combination with every-3-week versus weekly paclitaxel at doses of 60-100 mg/m². Response rates for weekly paclitaxel in these trials range from 56-84%, though with increased rates of peripheral neuropathy compared with every-3-week paclitaxel (Fountzilas et al. 2001; Gasparini et al. 2007; Seidman et al 2001; Gori et al. 2004; Perez et al 2005; Burris et al. 2004). For the purposes of the current study, low-to-intermediate doses of paclitaxel (65 mg/m² and 80 mg/m²) were chosen for administration on a weekly schedule in order to maximize clinical efficacy, as well as to allow for frequent monitoring with the ability to dose-reduce for toxicity on a weekly basis if needed.

In Cohort 1 of Part 1 investigating the every-3-week regimen of 2.4 mg/kg T-DM1 in combination with 65 mg/m² paclitaxel, two DLTs occurred: Grade 3 AST and ALT elevations and hospitalization due to dehydration secondary to nausea and vomiting. These DLTs precluded the testing of the weekly T-DM1 regimen starting at 2.0 mg/kg T-DM1 weekly.

On the basis of the current safety data observed in Part 1 of this study, Part 3 (dose escalation) will begin with a lower weekly starting dose of 1.2 mg/kg T-DM1 in combination with paclitaxel 65 mg/m² with dose escalation to 1.6 mg/kg and 2.0 mg/kg T-DM1 as tolerated.

The dose of pertuzumab for this study was chosen based on PK studies demonstrating similar pharmacokinetics observed across doses ranging from 2.0 mg/kg to 15.0 mg/kg (140 mg to 1050 mg for a 70 kg patient). A two-compartment model adequately described the concentration-time data with a terminal half-life of approximately 17 days for a typical patient. Based on these data, a dosing interval of 3 weeks was recommended for clinical studies.

In the Phase II studies, a loading dose of 840 mg (followed by 420 mg every 3 weeks) was capable of attaining steady-state trough and peak concentrations by the second cycle. Population PK modeling of data from Phase Ia and II studies supports the continued use of fixed non-weight based dosing in female patients.

### 2.3. Outcome Measures

### 2.3.1 Safety Outcome Measures

The safety and tolerability of T-DM1 and paclitaxel (and pertuzumab, if applicable) will be assessed using the following primary safety outcome measures:
   Highest tolerable doses of T-DM1 and paclitaxel when given in combination
   Frequency and nature of DLTs
   Incidence, nature, and severity of adverse events and serious adverse events
   Adverse events or changes in physical findings and clinical laboratory results during and following study treatment administration that result in dose modification, dose delay, or discontinuation of T-DM1, paclitaxel, and/or pertuzumab
   Change in cardiac function (i.e., LVEF, segmental wall abnormalities) as assessed by ECHO or MUGA scans

### 2.3.2 Pharmacokinetic and Pharmacodynamic Outcome Measures

The following PK parameters of T-DM1 and paclitaxel will be determined in all patients who receive study treatment (with the exception of patients in Dose Cohorts 5 and 9 [and Cohorts 5A, 5B, and 5C, if applicable]) using either non-compartmental and/or population methods, when appropriate, as data allow:
Serum concentrations of T-DM1 (conjugate), total trastuzumab (free and conjugated to DM1)
Plasma concentrations of DM1 Plasma concentrations of paclitaxel
   Total exposure (area under the concentration-time curve [AUC])
   Maximum concentration (Cmax)
   Minimum trough concentration (Cmin)
   Clearance
   Volume of distribution
   Terminal half-life
   Anti-therapeutic antibodies to T-DM1

### 2.3.3 Efficacy Outcome Measures

The following activity outcome measures will be assessed:
ORR based on investigator assessment using modified RECIST (v1.0)
PFS, defined as the time from the study treatment initiation to the first occurrence of disease progression or death on study (within 30 days of the last dose of study treatment) from any cause, as determined by investigator review of tumor assessments using modified RECIST (v1.0)
   For PD based on an increase in the sum of the longest diameter, a ≧20% increase in the sum of the longest diameter of target lesions from the smallest sum of the longest diameter and an absolute increase of ≧5 mm are required.
Duration of response, defined as the first occurrence of a documented objective response until the time of disease progression, as determined by investigator review of tumor assessments using modified RECIST (v1.0), or death on study (within 30 days of the last dose of study treatment) from any cause
Clinical benefit rate, defined as a clinical response or SD of ˜6 months duration as assessed by the investigator

### 2.3.4 Exploratory Outcome Measures

The following exploratory outcome measure will be assessed:
The relationship of HER2 status (determined by central laboratory testing) with ORR
   HER2 status will be determined by any or all of the following: immunohistochemistry (IHC), fluorescence in situ hybridization (FISH), RT-PCR, and/or assessment of the HER2-signaling pathway (including mutation analysis).
The safety of T-DM1 (and pertuzumab, if applicable) when administered to patients who have developed isolated brain metastases while on study treatment
Structural and functional changes in platelets prior to and following T-DM1 administration associated with thrombocytopenia
Internalization of T-DM1 into circulating platelets will be evaluated by immunofluorescence microscopy. Other structural and/or functional assays may also be performed.

### 2.4 Safety Plan

This is the first exposure of T-DM1 and paclitaxel (and pertuzumab, if applicable) administered in combination to humans; all patients will be monitored closely for toxicity. The potential safety issues anticipated in this trial, as well as measures intended to avoid or minimize such toxicities, are outlined in the following sections. All adverse events and serious adverse events will be recorded during the trial and up to 30 days after the last dose of T-DM1 and/or paclitaxel and/or pertuzumab. All adverse events will be monitored until they are assessed as stable or resolved or until another anti-cancer therapy is initiated. Patients will be followed for disease progression every two cycles until disease progression or the initiation of another anti-cancer therapy, whichever occurs sooner.

2.4.1 Risks Associated with T-DM1

T-DM1 has been administered to 52 patients in the Phase I study, and 112 patients in the Phase II study. Both of these studies are fully enrolled and have patients still on study; data collection is not yet complete.

Preliminary safety data are available for a total of 52 patients enrolled in the Phase I study: 24 patients on the every-3-week schedule and 28 patients on the weekly schedule. The most common Grade 1-2 toxicities reported were anemia, thrombocytopenia, and elevated liver enzymes. The Grade 1-2 toxicities were generally transient and reversible. Other potentially related Grade 1-2 adverse events included fatigue and headache.

Grade 3-4 adverse events observed with T-DM1 were Grade 4 thrombocytopenia (n=2), Grade 3 thrombocytopenia (n=1), and Grade 3 neutropenia (n=1). No dose-limiting cardiotoxicity has been observed thus far with T-DM1.

Preliminary data in 112 evaluable patients are also available from the Phase II study. The most common Grade 3-5 adverse events were thrombocytopenia (n=8) and hypokalemia (n=6). One patient died of respiratory failure within 2 weeks of stopping T-DM1 therapy; this was considered to be due to PD by the investigator. The five most common adverse events (of any grade) were fatigue, nausea, headache, constipation, and epistaxis.

2.4.2 Risks Associated with Paclitaxel

Risks associated with paclitaxel include myelosuppression, peripheral neuropathy, transaminase elevations, asymptomatic bradycardia, and hypersensitivity reactions. Please see the Taxol® Package Insert for more detailed information.

2.4.3 Risks Associated with Pertuzumab

Approximately 840 patients with cancer have been treated with pertuzumab in company-sponsored trials. Adverse events reported in pooled Phase II trials of single-agent pertuzumab (n=353) have generally been Grade 1-2 in severity and include diarrhea (58%), fatigue (32%), nausea (31%), abdominal pain (24%), vomiting (22%), anorexia (19%), and rash (17%).

Grade 3-4 adverse events have been less frequently reported, with the more frequent events being Grade 3 diarrhea (5%), Grade 3 vomiting (2%), and Grade 3 nausea (<1%). Asymptomatic decreases in LVEF have been reported as adverse events in 14% of patients and were mainly (−70%) Grade 1 events. LVEF declines of ≧10% to <50% in patients who had a baseline LVEF assessment and at least one post-baseline LVEF assessment were reported in 21/302 (7%) patients in completed Phase II single-agent studies and in 2/35 (6%) patients in the completed Phase Ib combination studies.

To date, symptomatic cardiac failure events have been reported in 4 of the 842 (0.5%) patients treated with pertuzumab across all studies. Two of these patients had MBC and had received prior anthracyclines and two patients had ovarian cancer patients, one of whom had cardiac risk factors (hypertension/hyperlipidemia).

### III. Examples

### 1. Materials and Methods

### 1.1 Patients

### 1.1.1 Inclusion Criteria

Patients must meet the following criteria to be eligible for study entry:
1. Signed Informed Consent Form
2. Age≧18 years
3. ECOG Performance Status 0-2
4. Locally advanced or metastatic breast cancer
   For the purposes of this study, locally advanced breast cancer is defined as unresectable local or regional disease.
5. HER2-positive breast cancer documented as fluorescence in situ hybridization (FISH)-positive, immunohistochemistry (IHC) 3+ or chromogenic in situ hybridization (CISH)-positive by local laboratory assessment
6. Confirmed availability (prior to enrollment) of tumor tissue blocks or 15-20 unstained tissue slides for confirmatory central laboratory HER2 status testing and other exploratory assessments. Tissue specimens must be submitted within 60 days after the first study treatment.
7. Prior trastuzumab in any line of therapy
8. No prior T-DM1 or pertuzumab therapy
9. Measurable or evaluable disease
Measurable disease is defined as at least one lesion ≧2 cm on computed tomography (CT) scan or ≧1 cm on spiral CT scan.
Evaluable disease is defined as clear radiographic or physical exam evidence of disease.
10. Cardiac ejection fraction≧50% by either ECHO or M UGA scan
11. Adequate hematologic and end organ function, defined by the following laboratory results obtained within 14 days prior to the first dose of study treatment:
   Absolute neutrophil count≧ 1500 cells/mm³
   Platelet count≧100,000 cells/mm³
   Hemoglobin≧9.0 g/dL
   Total bilirubin≦1.5×ULN
   SGOT (AST) and SGPT (ALT)≦2.5×ULN
   Serum creatinine≦1.5 mg/dL or creatinine clearance≧50 mL/min based on Cockcroft-Gault glomerular filtration rate (GFR) estimation:

(140−age)×(weight in kg)×(0.85 if female)/72×serum creatinine
12. For women of childbearing potential and men with partners of childbearing potential, agreement to use a highly effective form of non-hormonal contraception or two effective forms of non-hormonal contraception by the patient and/or partner, and to continue usage for the duration of the study treatment and for at least 6 months after the last dose of T-DM1 or pertuzumab. Male patients whose partners are pregnant should use condoms for the duration of the pregnancy.
13. Life expectancy≧90 days as assessed by the investigator

### 1.1.2 Exclusion Criteria

Patients who meet any of the following criteria will be excluded from study entry:
1. Fewer than 21 days since the last anti-tumor therapy, including chemotherapy, biologic, experimental, immune, radiotherapy for the treatment of breast cancer, with the following exceptions:
   Hormone-replacement therapy or oral contraceptives are allowed
   Palliative radiation therapy involving <_{—}25% of marrow-bearing bone is allowed if completed within >_{—}14 days prior to first study treatment
2. History of intolerance or hypersensitivity to trastuzumab and/or adverse events related to trastuzumab, murine proteins, or any of the excipients that resulted in trastuzumab being permanently discontinued
3. History of intolerance or hypersensitivity to paclitaxel and/or adverse events related to paclitaxel that resulted in paclitaxel being permanently discontinued
4. Peripheral neuropathy of Grade ≧2 per NCI CTCAE, Version 3.0, at the time of, or within 3 weeks prior to, the first study therapy
5. History of exposure to the cumulative doses of anthracyclines listed below. If another anthracycline or more than one anthracycline has been used, the cumulative dose must not exceed the equivalent of 500 mg/m² of doxorubicin.
   The Medical Monitor should be consulted if there are questions.
      Doxorubicin>500 mg/m² (or the equivalent dose of another anthracycline) Liposomal doxorubicin>900 mg/m² Epirubicin>720 mg/m²
6. History of clinically significant cardiac dysfunction, including:
   Current uncontrolled hypertension (systolic>150 mmHg and/or diastolic>100 mmHg), or unstable angina
   History of symptomatic CHF (Grade >3 by NCI CTCAE or Class >II by New York Heart Association [NYHA] criteria), or serious cardiac arrhythmia requiring treatment, with the exceptions of atrial fibrillation and paroxysmal supraventricular tachycardia
History of myocardial infarction within 6 months prior to first study treatment
7. Current known active infection with HIV, hepatitis B virus, or hepatitis C virus
8. Pregnancy or lactation
9. Current severe, uncontrolled systemic disease (e.g., clinically significant cardiovascular, pulmonary, or metabolic disease)
   Patients with severe dyspnea at rest, due to complications of advanced malignancy, or requiring supplementary oxygen therapy are also excluded.
10. Major surgical procedure or significant traumatic injury within 28 days prior to first study treatment, or anticipation of the need for major surgery during the course of study treatment
11. Symptomatic hypercalcemia requiring use of bisphosphonate therapy at the time of, or within 21 days of, the first study treatment
   Patients who are receiving bisphosphonate therapy specifically to prevent skeletal events and who do not have a history of clinically significant hypercalcemia are eligible.
12. Brain metastases that
   are: Untreated or
Progressive or
Have required any type of therapy (including radiation, surgery, or steroids) to control symptoms from brain metastases within 60 days prior to the first study treatment.
13. History of other malignancy within the last 5 years, except for appropriately treated carcinoma in situ of the cervix, basal cell carcinoma, or synchronous or subsequent HER2-positive breast cancer or other malignancy with a similar expected curative outcome

### 1.2 Method of Treatment Assignment

This is a single-arm, open-label study.

After written informed consent has been obtained and preliminary eligibility has been established, the study site will register patients with an IVRS at screening.

The IVRS will assign screening numbers for all patients. The investigator is responsible for assuring that results from all screening tests have been performed and that all eligibility criteria have been met. Once patient eligibility has been confirmed by the investigator, the study site will obtain the patient's identification number from the IVRS.

### 1.3 Study Treatment

### 1.3.1 Formulation, Administration, and Storage

a. T-DM1 Formulation

T-DM1 will be provided as a lyophilized formulation in a single-use vial in a colorless, 20-mL Type I glass vial closed by means of a FluroTec-coated stopper and an overseal with a flip-off cap. Each 20-mL vial contains enough product to deliver approximately 160 mg T-DM1. The contents of each vial must be dissolved in 8 mL Sterile Water for Injection (SWFI). The resulting liquid concentrate has a concentration of 20 mg/mL active ingredient. The lyophilized drug product, after reconstitution with 8.0 mL SWFI, contains 20 mg/mL T-DM1, 10 mM sodium succinate, pH 5.0, 60 mg/mL sucrose, and 0.02% (w/v) polysorbate 20. The reconstituted product contains no preservative and is intended for single use only.

All vials of T-DM1 should be handled by appropriately trained site staff wearing gloves and visually inspected upon receipt to ensure that they are intact without exterior contamination. Before administration (and after reconstitution for the lyophilized product), vials should be inspected to confirm they are clear and free of particulates.

The lyophilized product should be reconstituted using SWFI. Using a new syringe, add 8.0 mL SWFI and swirl gently until completely dissolved (do not shake vigorously). Inspect the vials to ensure the product is clear and free of particulates before proceeding. The seal of lyophilized product vials should be punctured once to introduce the 8.0 mL SWFI and once to remove the reconstituted product. As the reconstituted vials do not contain any preservative, they should be used within 1 hour of reconstitution. Vials that have been used for one patient may not be used for any other patient. Discard any vials containing unused product whose septum has been pierced, as the product does not contain preservative.

To prepare the infusion solution from the reconstituted lyophilized product, using a new syringe, remove the indicated volume of product from the vials, based on patient weight, and add to the IV bag. Gently invert the bag to mix the solution. Do not shake vigorously. Reconstituted T-DM1 is diluted into PVC or latex-free PVC-free polyolefin bags (PO) containing 0.45% or 0.9% Sodium Chloride injection (minimum volume of 250 mL). The use of PVC or PO bags containing 0.45% sodium chloride is preferred, and the use of 0.22 μm in-line filters is recommended. In cases wherein PVC or PO bags containing 0.9% sodium chloride are used, the use of 0.22 μm in-line filters is required. The diluted T-DM1 in infusion bags should be used immediately.

T-DM1 vials are to be refrigerated at 2° C.-8° C. (36° F.-46° F.) and should remain refrigerated until use. Do not freeze or shake vials. Protect the vials from light. T-DM1 vials should not be used beyond the expiration date provided by the manufacturer.

b. Pertuzumab Formulation

Pertuzumab is provided as a single use formulation containing 30 mg/mL pertuzumab in 20 mM L-histidine acetate (pH 6.0), 120 mM sucrose, and 0.02% polysorbate 20. Each 20 mL vial contains 420 mg pertuzumab (14.0 mL/vial).

Upon receipt of pertuzumab, vials are to be refrigerated at 2° C.-8° C. (36° F.-46° F.) until use. Pertuzumab vials should not be used beyond the expiration date provided by the manufacturer. Because the formulation does not contain a preservative, the vial seal may only be punctured once. Any remaining solution should be discarded. Vial contents should not be frozen.

The solution of pertuzumab for infusion diluted in PVC or non-PVC polyolefin bags containing 0.9% Sodium Chloride Injection, USP, may be stored at 2° C.-8° C. (36° F.-46° F.) for up to 24 hours prior to use. Diluted pertuzumab has been shown to be stable for up to 24 hours at room temperature (2° C.-25° C.). However, since diluted pertuzumab contains no preservative, the diluted solution should be stored refrigerated (2° C.-8° C.).

c. Paclitaxel Formulation

For details on paclitaxel formulation, see the Taxol® Package Insert.

### 1.3.2 Dosage

a. T-DM1 Dosage

T-DM1 will be administered every 3 weeks at a dose of 2.4, 3.0, or 3.6 mg/kg IV, or every week at a dose of 2.0 or 2.4 mg/kg IV during Part 1 (see Table 1) or every week at a dose of 1.2, 1.6, or 2.0 mg/kg IV during Part 3 (see Table 4). For the every-3-week schedule, any patient who has received a starting dose >2.4 mg/kg may be de-escalated to a T-DM1 dose as low as 2.4 mg/kg according to the dose-modification guidelines herein. For the weekly schedule, patients may be de-escalated to a T-DM1 dose as low as 2.0 mg/kg during Part I and as low as 1.2 mg/kg during Part 3 according to the dose-modification guidelines herein. Cycles are 21 days in length for both the every-3-week and weekly T-DM1 schedules.

The first infusion of T-DM1 will be administered over 90 (±10) minutes. If the first infusion of T-DM1 is well tolerated, subsequent infusions will be administered over 30-90 (±10) minutes. Vital signs should be assessed predose and postdose, and patients will be monitored for any untoward effects during the T-DM1 infusion and for at least 90 minutes after the first infusion and at least 30 minutes after infusions at subsequent cycles.

T-DM1 will be continued until progressive disease, unacceptable toxicity, initiation of another anti-cancer therapy, or the decision of the patient, investigator, or Sponsor. If T-DM1 is discontinued, the patient will discontinue all study treatment but will continue to be followed for 30 days for adverse events and serious adverse events and every 6 weeks for disease progression until the initiation of another anti-cancer therapy, patient- or investigator-initiated withdrawal from the study, or study termination by the Sponsor.

b. Paclitaxel Dosage

Paclitaxel will be administered no more frequently than weekly at a dose of 65 mg/m² or 80 mg/m² N. Paclitaxel should be administered weekly until disease progression or unacceptable toxicity. In order to gain an understanding of the tolerability of T-DM1 in conjunction with the chronic administration of paclitaxel, an attempt should be made to treat patients with a minimum of 12 consecutive weeks of paclitaxel.

Patients should be premedicated with dexamethasone, diphenhydramine, and cimetidine, or other H2 receptor antagonist 30-60 minutes prior to paclitaxel administration.

The first infusion of paclitaxel will be administered over 60 (±10) minutes. Vital signs should be assessed pre- and post-dose. Patients will be monitored for any untoward effects during the infusion and for at least 90 minutes after completion of the infusion. Subsequent infusion of paclitaxel will be administered over 30-60 (±10) minutes. Vital signs should be assessed pre- and post-dose, and patients will be monitored for any untoward effects during the infusion, and for at least 30 minutes after the completion of the infusion.

If paclitaxel is discontinued before disease progression, T-DM1 (and pertuzumab, if applicable) may be continued.

c. Pertuzumab Dosage

For patients enrolled into Dose Cohorts 5 and 9 (and Dose Cohorts 5A-5C, if applicable), pertuzumab will be administered at a loading dose of 840 mg IV on Day 1, Cycle 1, followed by 420 mg IV no more frequently than every 3 weeks in subsequent cycles.

The first infusion of pertuzumab will be administered over 60 (±10) minutes. Patients will be monitored for any adverse effects during the pertuzumab infusion and for at least 60 minutes after the first pertuzumab infusion and prior to the start of the T-DM1 infusion. If the first infusion of pertuzumab is tolerated, subsequent infusions will be also administered over 30-60 (±10) minutes. Vital signs will be assessed predose and postdose. Patients will be monitored for any untoward effects during the pertuzumab infusion for at least 30 minutes after each pertuzumab infusion at subsequent cycles.

Pertuzumab should be administered until disease progression or unacceptable toxicity. If pertuzumab is discontinued before disease progression, T-DM1 and paclitaxel may be continued.

1.3.3 Dose Modification

Patients should be assessed for toxicity prior to each dose of T-DM1 and/or paclitaxel (and/or pertuzumab, if applicable); dosing will occur only if the clinical assessment and laboratory test values are acceptable. In general, it is recommended that all T-DM1- and paclitaxel-related toxicities (and pertuzumab-related toxicities, if applicable) have resolved to Grade ≦1 or to the baseline grade prior to resuming therapy. Requirements for the recovery of specific toxicities are outlined below.

Administration of T-DM1 and/or paclitaxel (and/or pertuzumab, if applicable) may be delayed up to 21 days (42 days from last dose for the every-3-week regimen and 28 days from the last dose for the weekly regimen) to assess or treat adverse events. Any dose modification required during the DLT observation period will be classified as a DLT.

Patients should be re-evaluated weekly during any dose-delay period (or more frequently based on investigator discretion or if otherwise specified), whenever possible. Patients in whom significant toxicities have not recovered to Grade ≦1 or baseline grade at the time of their next scheduled dose may have their dose of T-DM1 and/or paclitaxel (and/or pertuzumab, if applicable) delayed for up to 21 days (42 days from last dose for the every-3-week regimen, and 28 days from the last dose for the weekly regimen). “Significant” and “related” will be based on the judgment of the investigator (in consultation with the Medical Monitor as needed). For example, alopecia, even if considered “related,” would most likely not be considered to be “significant.” Fatigue may or may not be considered either “related” or “significant.”

If toxicities do not resolve within the allowable dose delay period of 21 days (42 days from last dose for the every-3-week regimen, and 28 days from the last dose for the weekly regimen), the patient will discontinue study treatment but will continue to be followed for 30 days for adverse events and serious adverse events and every 6 weeks for disease progression until the initiation of another anti-cancer therapy, patient- or investigator-initiated withdrawal from the study, or study termination by the Sponsor.

For T-DM1, if retreatment criteria are met within the 21-day period, patients may receive the next scheduled dose of T-DM1 either at the prior dose level or at one dose level lower as described in Table 6. For paclitaxel, if retreatment criteria are met within the 21-day period, patients may receive the next scheduled dose of paclitaxel either at the previous dose level or at one dose level lower as described in Table 7.

Patients requiring a dose reduction will be de-escalated one dose level per the dose reduction guidelines until they are off study. No dose re-escalation will be allowed.

Patients who permanently discontinue paclitaxel (and/or pertuzumab, if applicable) may continue T-DM1. Patients in Parts 1 and 2 who permanently discontinue paclitaxel study treatment may be allowed to escalate the T-DM1 dose to 3.6 mg/kg every 3 weeks, per the clinical judgment of the investigator. Patients in Parts 3 and 4 who permanently discontinue paclitaxel study treatment will not be allowed to dose escalate the T-DM1 dose. Patients who discontinue T-DM1 will discontinue all study treatment but will continue to be followed for 30 days for adverse events and serious adverse events and every 6 weeks for disease progression until the initiation of another anti-cancer therapy, patient- or investigator-initiated withdrawal from the study, or study termination by the Sponsor.

TABLE 6
Dose Reduction for T-DM1 a

| | Every-3- | Part 1: | Part 3: |
|---|---|---|---|
| | Week | Weekly | Weekly |
| Dose Level | Schedule | Schedule | Schedule |
| For patients that | 3.6 mg/kg | NA | NA |
| permanently DC paclitaxel | | | |
| and re-escalate T-DM1 b | | | |
| 0 | 3.6 mg/kg | 2.4 mg/kg | 2.0 mg/kg |
| −1 | 3.0 mg/kg | 2.0 mg/kg | 1.6 mg/kg |
| −2 | 2.4 mg/kg | Off study | 1.2 mg/kg |
| Indication for further | Off study | Off study | Off study |
| dose reduction | | | |

DC = discontinued;
NA = not applicable.
a For dosing beyond Cycle 1. Dose reductions are not permitted during the dose-limiting toxicity observation period.
b Per clinical judgment of the investigator.

TABLE 7
Dose Reduction for Paclitaxel^{a}

| Dose Level | Paclitaxel Dose |
|---|---|
| 0 | 80 mg/m² |
| −1 | 65 mg/m² |
| Indication for further dose reduction | Off paclitaxel |

^{a}For dosing beyond Cycle 1. Dose reductions are not permitted during the dose-limiting toxicity observation period.

Additional dose delay and dose modification guidelines for specific T-DM1-, paclitaxel-, and/or pertuzumab-related toxicities, as described below, are to serve as guidelines to allow ongoing treatment for patients experiencing clinical benefit while ensuring patient safety.

a. T-DM1 and Paclitaxel Dose Modification for Hematologic, Hepatic, and Neurologic Toxicity

For hematologic, hepatic, and neurologic toxicities, which are common to both T-DM1 and paclitaxel, the decision of whether to dose-reduce and/or discontinue T-DM1, paclitaxel, or both will be at the discretion of the investigator after careful assessment and discussion of the risks versus benefits with the patient.

For example, if a patient experiences both thrombocytopenia and neutropenia, this may represent primarily toxicity of paclitaxel rather than T-DM1, requiring dose modification of paclitaxel alone. However, if a patient were to experience an isolated, transient thrombocytopenia, this may represent primarily toxicity of T-DM1, requiring dose modification of T-DM1 alone. In certain cases, the investigator may decide that dose reduction of both agents simultaneously is warranted. Guidelines are outlined below, and investigators may contact the Medical Monitor for further guidance in specific cases as needed.

T-DM1 and Paclitaxel Dose Modification for Hematologic Toxicity

The investigator or study staff should contact the Medical Monitor immediately for the first occurrence of any Grade 3 or the first occurrence of any Grade 4 thrombocytopenia to discuss additional work-up and follow-up of the thrombocytopenia. Patients who experience Grade 2 or Grade 3 thrombocytopenia should have the next dose of T-DM1 held until the platelet count has recovered to Grade ≦1 or to the baseline grade. Patients who experience a first Grade 4 thrombocytopenia event may, after adequate recovery to a platelet count of Grade ≦1 or to the baseline grade, continue treatment with dose reduction of T-DM1 and/or paclitaxel to one dose level lower. Patients who experience a second Grade 4 thrombocytopenia event may, after adequate recovery as defined above, continue treatment with further dose reductions of T-DM1 and/or paclitaxel to one dose level lower than the dose at which the second event was experienced (see Table 6 and/or Table 7). No re-escalation of the T-DM1 or paclitaxel dose will be allowed. Patients who require a dose reduction for T-DM1 below 2.4 mg/kg (for the every-3-week schedule) or 2.0 mg/kg (for the weekly schedule during Part 1) or 1.2 mg/kg (for the weekly schedule during Part 3) will discontinue all study treatment but will continue to be followed for 30 days for adverse events and serious adverse events and every 6 weeks for disease progression until the initiation of another anti-cancer therapy, patient- or investigator initiated withdrawal from the study, or study termination by the Sponsor. A dose delay of 21 days is permitted (42 days from last dose for the every-3-week regimen and 28 days from the last dose for the weekly regimen).

Patients who experience a Grade 4 hematologic adverse event of neutropenia and/or thrombocytopenia should be checked twice per week (or more often based on investigator discretion) for the recovery of their neutrophil and/or platelet count. Patients who experience a similar Grade 3 event should be checked weekly for the recovery of their neutrophil and/or platelet counts. If a patient's neutrophil and/or platelet count does not recover to baseline or Grade ≦1 within the allowable delay of 21 days, the patient will discontinue all study treatment but will continue to be followed for 30 days for adverse events and serious adverse events and every 6 weeks for disease progression until the initiation of another anti-cancer therapy, patient- or investigator-initiated withdrawal from the study, or study termination by the Sponsor.

T-DM1 and/or Paclitaxel Dose Modification for Hepatotoxicity

Patients who experience a first Grade ≧3 hepatic event (increased elevations of transaminases and/or total bilirubin) may, after adequate recovery to Grade ≦1 or baseline, continue treatment with a dose reduction of T-DM1 and/or paclitaxel to one dose level lower in subsequent treatment cycles. Patients who experience a second Grade ≧3 hepatic event may, after adequate recovery as defined above, continue treatment with further dose reduction of T-DM1 and/or paclitaxel to one level lower than the dose level at which the second event was experienced. A dose delay of 21 days is permitted (42 days from last dose for the every-3-week regimen and 28 days from the last dose for the weekly regimen). Patients who require a dose reduction for T-DM1 below 2.4 mg/kg (for the every-3-week schedule) or 2.0 mg/kg (for the weekly schedule) or 1.2 mg/kg (for the weekly schedule during Part 3) will discontinue all study treatment but will continue to be followed for 30 days for adverse events and serious adverse events and every 6 weeks for disease progression until the initiation of another anti-cancer therapy, patient- or investigator-initiated withdrawal from the study, or study termination by the Sponsor. A dose delay of 21 days is permitted.

In general, it is recommended, but not required, that dose reduction of paclitaxel be attempted first in order to allow for maximal exposure to T-DM1 in patients with ongoing clinical benefit. No re-escalation of the T-DM1 or paclitaxel dose will be allowed.

Patients who experience a Grade ≧3 hepatic adverse event should have their liver enzymes and/or total bilirubin checked twice per week until they are stable or until recovery to Grade ≦2 (or 25% above baseline in patients with hepatic dysfunction as a result of liver or bone metastases). If a patient's liver enzymes do not recover to baseline or Grade ≦1 (or baseline) within the allowable dose delay of 21 days, the patient will discontinue all study treatment but will continue to be followed for 30 days for adverse events and serious adverse events and every 6 weeks for disease progression until the initiation of another anti-cancer therapy, patient- or investigator-initiated withdrawal from the study, or study termination by the Sponsor.

T-DM1 and/or Paclitaxel Dose Modification for Neurotoxicity

For patients who experience Grade >_{—}3 neuropathy, a dose delay of up to 21 days (42 days from last dose for the every-3-week regimen and 28 days from last dose for the weekly regimen) is permitted for T-DM1 and/or paclitaxel. After adequate recovery to Grade ≦1, the investigator may consider decreasing the dose of T-DM1 and/or paclitaxel by one dose level. It is recommended, but not required, that dose reduction of paclitaxel be attempted first in order to allow for maximal exposure to T-DM1 in patients with ongoing clinical benefit.

No re-escalation of the T-DM1 or paclitaxel dose will be allowed. If neuropathy does not resolve to Grade ≦1 within 42 days from last dose for the every-3-week regimen, and 28 days from the last dose for the weekly regimen, the patient will discontinue all study treatment but will continue to be followed for 30 days for adverse events and serious adverse events and every 6 weeks for disease progression until the initiation of another anti-cancer therapy, patient- or investigator-initiated withdrawal from the study, or study termination by the Sponsor.

b. T-DM1 and Pertuzumab Dose Modification for Other Specific Toxicities T-DM1 and Pertuzumab Dose Modification for Infusion Reactions

Infusion of T-DM1 (and/or pertuzumab) should be interrupted for patients who develop dyspnea or clinically significant hypotension. The infusion should be slowed to ˜50% or interrupted for patients who experience any other infusion-related symptoms. When the patient's symptoms have completely resolved, the infusion may be continued at ˜50% of the rate prior to the reaction and increased in 50% increments every 30 minutes as tolerated. Infusions may be restarted at the full rate during the next cycle. Supportive care with oxygen, beta agonists, antihistamines, antipyretics, or corticosteroids may be used as appropriate at the investigator's discretion. Premedication with corticosteroids, antihistamines, and antipyretics may be used before subsequent infusions of T-DM1 at the investigator's discretion. Patients should be monitored until complete resolution of symptoms. Patients who experience a Grade ≧3 hypersensitivity reaction or acute respiratory distress syndrome will be discontinued from the study. Continued treatment with T-DM1 and paclitaxel may be considered in selected patients with ongoing clinical benefit if the hypersensitivity reaction can be clearly attributed to pertuzumab after careful assessment and discussion of the risks versus benefits with the patient.

T-DM1 and Pertuzumab Dose Modification for Cardiotoxicity

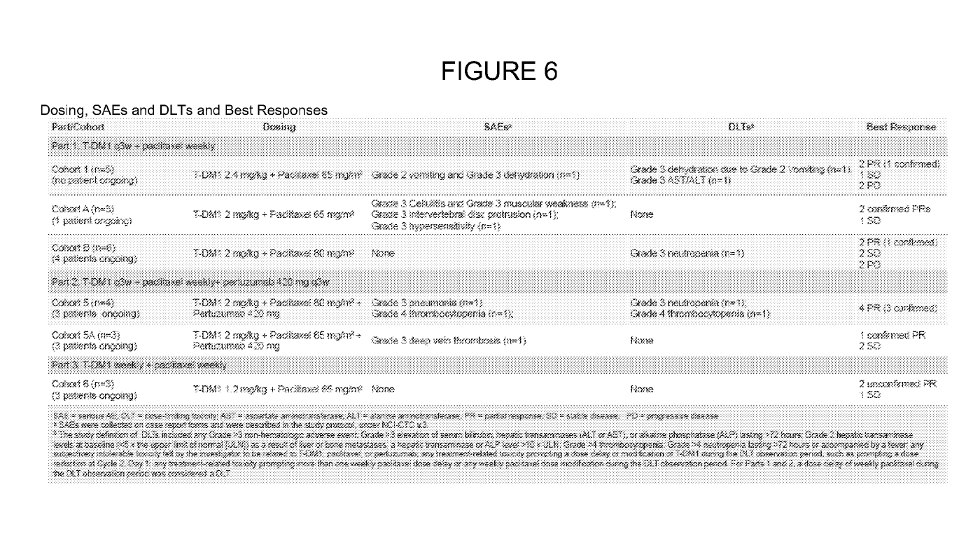

Patients without a significant cardiac history and with an LVEF of 50% as determined by ECHO or MUGA scan are eligible for study participation. Ejection fractions will be monitored at baseline, at the end of Cycle 2, and every three cycles thereafter. Although cardiac toxicity has not yet been observed in patients receiving T-DM1, cardiac toxicity could also occur in association with T-DM1 therapy based on the known toxicities of trastuzumab. Patients with confirmed symptomatic CHF (Grade ≧3 left ventricular systolic dysfunction as defined by NCI CTCAE, Version 3.0) will discontinue T-DM1 (and pertuzumab, if applicable). CHF should be treated and monitored according to standard medical practice. FIG. 6 summarizes the management of T-DM1 (and pertuzumab, if applicable) based on LVEF assessments in asymptomatic patients. Study treatment continuation with T-DM1 (and pertuzumab, if applicable) may be considered for asymptomatic patients with ongoing clinical benefit and depending on the safety data from the ongoing T-DM1 (and pertuzumab, if applicable) trials after careful assessment and discussion of the risk versus benefit with the patient and with approval of the Medical Monitor.

Local review of ECHO and MUGA scans will be used for clinical decisions during the trial. LVEF will be monitored regularly according to the schedule of assessments at screening, at the end of Cycle 2, and every three cycles throughout the treatment period. If an investigator is concerned that an adverse event may be related to cardiac dysfunction, additional LVEF measurement(s) may be performed.

T-DM1 Dose Modification for Other Toxicities

If a patient develops any other Grade 3 or 4 toxicity thought to be related specifically to T-DM1, T-DM1 should be held until symptoms resolve to Grade ≦1 or to the baseline grade. When treatment is resumed, the T-DM1 dose should be modified according to the guidelines in Table 6. If Grade ≧3 toxicity persists for more than 21 days (42 days from last dose for the every-3-week regimen and 28 days from last dose for the weekly regimen) or recurs after dose reduction, the patient will discontinue T-DM1 treatment and all other study treatment but will continue to be followed for 30 days for adverse events and serious adverse events and every 6 weeks for disease progression until the initiation of another anti-cancer therapy, patient- or investigator-initiated withdrawal from the study, or study termination by the Sponsor.

Pertuzumab Dose Modification for Other Toxicities

No dose modification for pertuzumab will be allowed in this study. Patients who experience any other Grade 3 or 4 toxicity thought to be due to pertuzumab will discontinue pertuzumab permanently. Patients who discontinue pertuzumab may continue to receive treatment with T-DM1 and paclitaxel.

Paclitaxel Dose Modification for Anaphylaxis/Hypersensitivity

Paclitaxel infusion should be interrupted for patients who develop dyspnea or clinically significant hypotension. Supportive care with oxygen, beta agonists, antihistamines, antipyretics, corticosteroids, or epinephrine may be used as appropriate at the investigator's discretion. Patients who experience a Grade ≧3 hypersensitivity reaction or acute respiratory distress syndrome will be discontinued from study treatment. Continued treatment with T-DM1 (and pertuzumab, if applicable) may be considered in individual cases if the hypersensitivity reaction can be clearly attributed to paclitaxel (e.g., the reaction occurs on Day 8 or Day 15 in a patient on the every-3-week schedule, or the reaction occurs after paclitaxel administration but prior to the administration of another study drug) after careful assessment and discussion of the risks versus benefits with the patient.

Paclitaxel Dose Modification for Other Toxicities

If a patient develops any other Grade 3 or 4 toxicity thought to be related to paclitaxel, paclitaxel should be held until symptoms resolve to Grade ≦1 or to baseline grade. When treatment is resumed, the paclitaxel dose should be reduced permanently to 65 mg/m² or discontinued if the patient was already receiving 65 mg/m², as described in Table 7. If Grade 3 toxicity persists for more than 21 days (42 days from last dose for the every-3-week regimen and 28 days from last dose for the weekly regimen) or recurs after dose reduction, the patient will permanently discontinue paclitaxel treatment.

### 1.4 Assay Methods

### 1.4.1 Drug and Antibody Levels

Serum samples will be analyzed for T-DM1, total trastuzumab, and HER2 extracellular domain (ECD) levels using a validated ELISA method. Lithium-heparin plasma samples will be assayed to measure DM1 using a validated liquid chromatography electrospray tandem mass spectrometry (LC—MS/MS) method. Serum samples will also be assayed for anti-therapeutic antibodies to T-DM1 in a bridging antibody electrochemiluminescence assay. Paclitaxel concentrations will be determined in plasma samples obtained in this study using an appropriate validated LC—MS/MS method.

### 1.4.2 Tissue Samples

The availability of tumor tissue blocks or 15-20 unstained tissue slides will be confirmed at screening for subsequent confirmatory central laboratory HER2 status testing and other exploratory assessments, including levels of expression of HER family proteins, the HER2 signaling pathway and other related pathways. Analysis may also include mutation status of genes in the HER2 signaling pathway.

HER2 gene amplification will be assessed on archival tumor material using the Abbott PathVyision HER2 FISH kit, and HER2 protein overexpression by IHC will be performed using the DAKO HercepTest kit according to the manufacturer's instructions.

If the availability of tumor tissue blocks or unstained slides is confirmed, these may be collected after the patient has enrolled in the study. Tissue specimens must be submitted within 60 days after the first study treatment.

Paraffin block tumor tissue and/or additional slides from patients will be collected and sent to a specialty laboratory for analysis. Some samples may be enriched for tumor content by macro dissection of histologically identifiable tumor. RNA will be extracted, and quantitative RT-PCR for HER family receptors and/or ligands and a reference gene will be performed using a standard platform (e.g., LightCycler or TaqMan).

Other biomarker assessments that may be related to the method of action of T-DM1, HER2 signaling, or breast cancer may be performed in this study on the archival tissue collected at study entry.

### 1.4.3 Optional Research: Platelet Analysis

The objective of this exploratory analysis is to further characterize the etiology of T-DM1—associated thrombocytopenia. The patients' participation in this analysis is optional and pertains only to patients enrolled in Part 3 at the DFCI.

Approximately 1 mL of blood will be collected in tubes containing Aster-Jandl anticoagulant prior to and following T-DM1 administration at Cycle 1 and Cycle 2.

Internalization of T-DM1 into circulating platelets will be assessed by immunofluorescence microscopy. Other structural and/or functional assays may additionally be performed.

### 1.5 Statistical Methods

The final analysis will be based on patient data collected through study discontinuation by Genentech until all patients enrolled have discontinued study treatment or until the last patient in the study has completed 12 months of study treatment, whichever occurs first. Analyses will include all efficacy-evaluable patients, defined as all patients who receive any amount of study treatment and who have had at least one follow-up tumor assessment.

Descriptive summaries of continuous data will be presented for the group mean, standard deviation, median, minimum, maximum, and sample size.

Descriptive summaries of discrete data will be presented for the number of patients and the incidence, as a frequency and as a percentage.

### 1.5.1 Analysis of the Conduct of the Study

The number of enrolled patients will be tabulated by study site, dose group, and overall. Eligibility exceptions and protocol deviations will be tabulated by dose group and overall. Patients' disposition and reasons for premature discontinuation will be tabulated by dose group and overall.

### 1.5.2 Safety Analyses

Safety will be assessed through summaries of deaths, DLTs, adverse events, serious adverse events, changes in laboratory test results, dose delays, dose reductions, and reasons for study treatment discontinuation. All patients who receive any amount of study treatment will be included in the safety analysis, and the data will be summarized according to the dose received. Verbatim descriptions of adverse events will be mapped to thesaurus terms.

All collected adverse event data will be listed by study site, dose received, patient, and cycle. All adverse events occurring on or after the first treatment will be summarized by mapped term, appropriate thesaurus levels, and NCI CTCAE (Version 3.0) toxicity grade. In addition, all serious adverse events, including deaths, will be listed separately and summarized.

Occurrence of DLTs experienced during the DLT observation period will be summarized by dose group based on data from patients evaluable for dose-escalation decisions and/or MTD assessment. Deaths reported during the study treatment period and those reported during follow-up after patient treatment discontinuation will be summarized.

Laboratory data will be listed, with values outside of normal ranges identified. For each patient, laboratory data will be plotted over time. Additionally, laboratory data will be summarized by grade using the NCI CTCAE (Version 3.0) toxicity grade. Changes in LVEF over time will be summarized and listed by dose group and scheduled measurement time.

### 1.5.3 Pharmacokinetic and Pharmacodynamic Analyses

In this study, samples for the determination of plasma paclitaxel, plasma DM1, serum T-DM1, and serum total trastuzumab will be collected in all patients (with the exception of those in Dose Cohorts 5 and 9 [and Dose Cohorts 5A-5C, if applicable]). For paclitaxel and T-DM1, the sampling will allow the determination of the total exposure (AUCO-t), Cmax, and Cmin. Other pharmacokinetic parameters may be determined as data allow.

The frequent sampling schedule following the single dose of paclitaxel alone on Cycle 1, Day—1 will allow determination of the pharmacokinetics of paclitaxel in the absence of T-DM1. Sparse collection of T-DM1 serum concentrations, total trastuzumab, and DM1 concentrations following the administration of T-DM1 at Cycle 1, Day 1 and following the administration of paclitaxel and T-DM1 at Cycle 2, Day 1, will allow determination of the potential impact of paclitaxel on T-DM1 pharmacokinetics. This will also allow comparison with previous historical observations in single-agent T-DM1 trials.

Paclitaxel, T-DM1, and total trastuzumab descriptive statistics, including mean and median trough and peak values, will be summarized by patient and dose group. For unconjugated DM1, levels will be summarized for each evaluable patient at each timepoint. AUC, Cmax, clearance, volume of distribution, and half-life will be calculated where possible. PK parameters will be determined using best available techniques that can be applied to all available data. Population PK and noncom partmental PK methods will be considered.

### 1.5.4 Efficacy Analyses

Analyses will include all efficacy-evaluable patients, defined as all patients who receive any amount of study treatment and who have had at least one follow-up tumor assessment.

Best ORR, PFS, duration of objective response, and clinical benefit rate will be listed by dose group. Any patient with insufficient data for response determination will be classified as a nonresponder. For patients who do not have documented progressive disease or death on study, PFS and duration of response will be censored at the day of the last tumor assessment.

Objective Response Rate

Investigator-assessed objective response is defined as a complete or partial response (using modified RECIST, v1.0) determined on two consecutive occasions ≧4 weeks apart. An estimate of the objective response rate will be computed as well as the corresponding 95% confidence interval.

Progression-Free Survival

Kaplan-Meier estimate of median PFS will be reported as well as the corresponding 95% confidence interval. Patients without disease progression or death will be censored at the time of the last tumor assessment.

Duration of Objective Response

Duration of objective response will be assessed for patients with an objective response. Kaplan-Meier estimate of median duration of objective response will be reported as well as the corresponding 95% confidence interval. Patients without disease progression or death will be censored at the time of the last tumor assessment.

Clinical Benefit Rate

Clinical benefit rate is defined as CR, PR, or SD of ˜6 months duration as assessed by the investigator. An estimate of the clinical benefit rate will be computed as well as the corresponding 95% confidence interval.

### 1.5.5 Exploratory Analyses

All data for biologic markers, including HER2 status (determined by central laboratory testing), will be listed by dose group and response status. Adverse events will be summarized separately for the subgroup of patients who have developed isolated brain metastases while on study treatment.

### 1.5.6 Handling of Missing Data

For objective response, patients without a valid post-baseline tumor assessment will be counted as nonresponders. For duration of response and PFS, data from patients who are lost to follow-up will be treated as censored on the last date the patient was known to be progression-free. Data for patients without post-treatment tumor assessment or death will be censored at the date of the treatment initiation plus 1 day.

### 1.5.7 Determination of Sample Size

Approximately 36 patients will be enrolled in this trial. The final sample size will be determined by the dose-escalation rules described in the Study Design Section.

### 1.6 Data Quality Assurance

The data will be collected via Electronic Data Capture (EDC) using eCRFs. The site will be responsible for data entry into the EDC system. In the event of discrepant data, the CRO will request data clarification from the sites, which the sites will resolve electronically in the EDC system. The CRO will be responsible for the data management of this trial, including quality checking of the data.

Genentech will perform oversight of the data management of this trial. Genentech will produce an EDC Study Specification document that describes the quality checking to be performed on the data. Central Laboratory data and other electronic data will be sent directly to Genentech, using Genentech's standard procedures to handle and process the electronic transfer of these data.

eCRFs and correction documentation will be maintained in the EDC system's audit trail. System backups for data stored at Genentech and records retention for the study data will be consistent with Genentech's standard procedures.

### 2. Assessment of Safety

### 2.1 Safety Parameters and Definitions

Safety assessments will consist of monitoring and recording protocol-defined adverse events (AEs) and serious adverse events (SAEs); measurement of protocol-specified hematology, clinical chemistry, and urinalysis variables; measurement of protocol-specified vital signs; and other protocol-specified tests that are deemed critical to the safety evaluation of the study treatment(s).

Genentech or its designee is responsible for reporting relevant SAEs to the Competent Authority, other applicable regulatory authorities, ECs, and participating investigators, in accordance with ICH guidelines, FDA regulations, European Clinical Trials Directive (Directive 2001/20/EC), and/or local regulatory requirements.

Genentech or its designee is responsible for reporting unexpected fatal or life-threatening events associated with the use of the study drug to the regulatory agencies and competent authorities by telephone or fax within 7 calendar days after being notified of the event. Genentech or its designee will report other relevant SAEs associated with the use of the study medication to the appropriate competent authorities (according to local guidelines), investigators, and central

IRBs/ECs (except in the United States where investigators are responsible for reporting to their IRBs per local requirements) by a written safety report within 15 calendar days of notification.

### 2.1.1 Adverse Event

An AE is any unfavorable and unintended sign, symptom, or disease temporally associated with the use of an investigational medicinal product (IMP)
or other protocol-imposed intervention, regardless of attribution.

This includes the following:
AEs not previously observed in the patient that emerge during the protocol-specified AE reporting period, including signs or symptoms associated with breast cancer that were not present prior to the AE reporting period
Complications that occur as a result of protocol-mandated interventions (e.g., invasive procedures such as biopsies)
AEs that occur prior to assignment of study treatment that are related to a protocol-mandated intervention (e.g., invasive procedures such as biopsies, medication washout, or no treatment run-in).
Preexisting medical conditions (other than breast cancer), judged by the investigator to have worsened in severity or frequency or changed in character during the protocol-specified AE reporting period

### 2.1.2 Serious Adverse Event

An SAE is any AE that is any of the following:
Fatal (i.e., the AE actually causes or leads to death)
Life threatening (i.e., the AE, in the view of the investigator, places the patient at immediate risk of death)
Requires or prolongs inpatient hospitalization
Results in persistent or significant disability/incapacity (i.e., the AE results in substantial disruption of the patient's ability to conduct normal life functions)
A congenital anomaly/birth defect in a neonate/infant born to a mother exposed to the investigational product(s)
Considered a significant medical event by the investigator (e.g., may jeopardize the patient or may require medical/surgical intervention to prevent one of the outcomes listed above)

All AEs that do not meet any of the criteria for serious should be regarded as nonserious AEs. The terms “severe” and “serious” are not synonymous. Severity refers to the intensity of an AE (as in mild, moderate, or severe pain); the event itself may be of relatively minor medical significance (such as severe headache).

“Serious” is a regulatory definition and is based on patient or event outcome or action criteria usually associated with events that pose a threat to a patient's life or vital functions. Seriousness (not severity) serves as the guide for defining regulatory reporting obligations.

Severity and seriousness should be independently assessed when recording AEs and SAEs on the eCRF.

### 2.1.3 Protocol-Defined Events of Special Interest/Nonserious Expedited Adverse Events

The following are events of special interest for patients treated with T-DM1, paclitaxel (and pertuzumab, if applicable) and will need to be reported to the Sponsor expeditiously, irrespective of regulatory seriousness criteria or causality:
Symptomatic CHF (Grade ≧3 left ventricular systolic dysfunction)
Asymptomatic declines in LVEF that lead to permanent discontinuation of study drug: a confirmed LVEF of <40% or an LVEF of ≦45% with a ≧10% change from baseline as assessed by ECHO or MUGA scan

In addition to reporting adverse event grading severity, events of symptomatic LVEF decline or CHF (left ventricular systolic dysfunction Grade ≦3) should also be graded using the NYHA classification.

### 2.2 Assessment of Severity and Causality of Adverse Events

Investigators will seek information on AEs and SAEs at each patient contact. All AEs and SAEs, whether reported by the patient or noted by authorized study personnel, will be recorded in the patient's medical record and on the AE eCRF page.

For each AE and SAE recorded on the applicable eCRF, the investigator will make an assessment of seriousness (seriousness criteria provided herein), severity, and causality. Table 8 provides guidance for grading AE severity and Table 9 provides guidance for assessing the causal relationship to the investigational product(s).

The AE grading (severity) scale found in the NCI CTCAE (Version 3.0) will be used for AE reporting.

TABLE 8
Adverse Event Grading (Severity) Scale

| Grade | Severity | Alternate Description^{a} |
|---|---|---|
| 1 | Mild (apply event-specific | Transient or mild discomfort (<48 |
| | NCI CTCAE grading criteria) | hours); no interference |
| | | with the patient''s daily activities; no |
| | | medical intervention/therapy required |
| 2 | Moderate (apply event-specific | Mild to moderate interference with the |
| | NCI CTCAE grading criteria) | patient''s daily activities; no or minimal |
| | | medical intervention/therapy required |
| 3 | Severe (apply event-specific | Considerable interference with the |
| | NCI CTCAE grading criteria) | patient's daily activities; medical |
| | | intervention/therapy required; |
| | | hospitalization possible |
| 4 | Very severe, life threatening, | Extreme limitation in activity; significant |
| | or disabling (apply event-specific | medical intervention/therapy required, |
| | NCI CTCAE grading criteria) | hospitalization probable |
| 5 | Death related to AE | |

The NCI CTCAE v3.0 can be found: http://ctep.cancer.gov/reporting/ctc_v30.html.
Note:
Regardless of severity, some events may also meet regulatory serious criteria. Refer to definitions of an SAE herein.
^{a}Use these alternative definitions for Grade 1, 2, 3, and 4 events when the observed or reported AE is not in the NCI CTCAE listing.

### 3. Results

24 patients were enrolled at 4 sites, with 14 still on study. All patients had received prior trastuzumab; 19 had received prior lapatinib. 23 had received a prior taxane (Table 9). Patients had received a median of ten prior agents (not including hormonal therapies). All patients were female.

TABLE 9
Demographics and Baseline Characteristics,
Including Prior Therapies

| | All Patients (n = 24) | |
|---|---|---|
| Median Age, years (range) | 54 | (35-77) |
| Race, n (%) | | |
| White | 22 | (91.7) |
| Asian | 2 | (8.3) |
| ECOG performance status, n (%) | | |
| 0 | 11 | (45.8) |
| 1 | 12 | (50.0) |
| 2 | 1 | (4.2) |
| Radiotherapy | 24 | (100.0) |
| Surgery | 24 | (100.0) |
| Prior systemic therapies,* n (%) | 24 | (100.0) |
| Trastuzumab | 24 | (100.0) |
| Chemotherapy | 24 | (100.0) |
| Taxane | 23 | (95.8) |
| Lapatinib | 19 | (79.2) |
| Hormonal | 14 | (58.3) |
| Other | 13 | (54.2) |
| Median total number of prior systemic | 10 | (5-21) |
| agents** (range) | | |
| Median number of prior systemic agents in | 7 | (0-18) |
| metastatic setting** (range) | | |
| Median time since metastatic diagnosis, | 38.2 | (9-111) |
| months (range) | | |

*Includes 12 patients who received another biologic (bevacizumab), experimental therapies, other targeted therapies, or other therapies.
**Does not include hormonal therapy

### DLT Assessment (FIG. 6)

Part 1: The MTD for T-DM1 in combination with weekly paclitaxel 80 mg/m² was established at 2 mg/kg q3w.
   There were two DLTs in 5 patients dosed at T-DM1 2.4 mg/kg q3w+paclitaxel 65 mg/m² weekly.
   One patient was hospitalized for grade 3 dehydration secondary to nausea and vomiting
   One patient developed grade 3 elevations of alanine aminotransferase (ALT) and aspartate aminotransferase (AST).
   There was one DLT of grade 3 neutropenia among 6 patients dosed at T-DM1 2 mg/kg q3w+paclitaxel 80 mg/m² weekly
Part 2: Pertuzumab was added to the MTD from Part 1. Two DLTs (grade 3 neutropenia and grade 4 thrombocytopenia) were observed among 4 patients. The MTD was established when nc DLTs were observed in 3 patients dosed at 2 mg/kg T-DM1q3w+paclitaxel 65 mg/m² weekly+pertuzumab 420 mg q3w.
Part 3: No DLTs have been observed in 3 patients enrolled at T-DM1 weekly (starting dose 1.2 mg/kg)+65 mg/m² paclitaxel.
All SAEs and DLTs were grade 3 except for grade 4 thrombocytopenia in a patient receiving T-DM1, paclitaxel and pertuzumab.

### Responses (FIG. 6)

Among the 24 evaluable patients in the cohorts, there have been 13 partial responses (8 confirmed). Of the 5 unconfirmed partial responses, 1 is unconfirmed due to disease progression, and 4 are unconfirmed due to insufficient follow-up (<12 weeks).
Among patients with a confirmed partial response, the duration of response ranged from 1.41 (ongoing) to 7.06 months.
Three patients had isolated CNS disease progression. Two of these patients continued on study treatment and one discontinued the study to be treated with trastuzumab. Of the 2 patients who continued on study, one patient had received 9 cycles of treatment prior to isolated CNS progression and received 5 additional cycles until further disease progression in the CNS. The second patient received 12 cycles of treatment before isolated CNS progression, and received 2 additional cycles until systemic disease progression.

### Safety (Tables 10 and 11)

Of 24 patients, 12 experienced at least one grade ≧3 AE.
No grade 5 AEs have been reported, and one grade 4 AE was observed: thrombocytopenia in a patient in Part 2, Cohort 5.
The grade 3 AEs occurring in >1 patient were neutropenia (n=4), anemia (n=3), thrombocytopenia (n=2), and ALT increased (n=2).
The most common AEs of any grade were fatigue (79.2%), peripheral neuropathy (75%) and alopecia (41.7%).

TABLE 10
Adverse Events (any grade) Occurring
in >20% of Patients (n = 24)

| Adverse Event, n (%) | n (%) |
|---|---|
| Fatigue | 19 (79.2) |
| Peripheral Neuropathy | 18 (75.0) |
| Alopecia | 10 (41.7) |
| Diarrhea | 9 (37.5) |
| Arthralgia | 8 (33.3) |
| Epistaxis | 8 (33.3) |
| Nausea | 8 (33.3) |
| Constipation | 7 (29.2) |
| Anemia | 6 (25.0) |
| Dry Eye | 5 (20.8) |
| Dyspepsia | 5 (20.8) |
| Dyspnea | 5 (20.8) |
| Headache | 5 (20.8) |

TABLE 11
All Grade ≧AEs*(n = 24)

| Adverse Event, n (%) | Number of patients, n (%) |
|---|---|
| Neutropenia | 4 (16.7) |
| Anemia | 3 (12.5) |
| ALT increased | 2 (8.3) |
| Thrombocytopenia | 2 (8.3) |

The following grade ≧3 events occurred in 1 (4.2%) patient:
AST increased, cellulitis, dehydration, fatigue, hemoglobin decreased, hypersensitivity, hypokalemia, hypotension, intervertebral disc protrusion, muscular weakness, pneumonia, polydipsia
*All events were grade 3 except for one grade 4 thrombocytopenia event.

### Study Discontinuation

Ten patients had discontinued from the study as of the data cutoff. Reasons for discontinuation included disease progression per RECIST criteria (n=7), physician's decision (clinical disease progression not documented by RECIST) (n=1), patient decision (n=1), and AE (grade 2 thrombocytopenia) (n=1).

Patients with ongoing clinical benefit who discontinued paclitaxel due to toxicity were permitted to continue single agent T-DM1 (in Parts 1 & 3) or T-DM1/pertuzumab (in Part 2).

### Paclitaxel Dose Reduction/Discontinuation

Four patients had paclitaxel dose reductions to 65 mg/m², two in Part 1 (due to neutropenia and anemia, respectively) and two in Part 2 (due to elevated ALT/AST and pneumonia, respectively).

Eight patients had paclitaxel discontinued but continued on study drug(s): 3 for peripheral neuropathy, and 1 each for anemia, neutropenia, thrombocytopenia, transaminase elevation, and hypersensitivity.

### Pharmacokinetics

PK assessments showed that the PK of T-DM1 in the presence of paclitaxel in TDM4652g is comparable with historical phase I data (see 2010 SABCS poster #P3-14-22 by Lu et al. for further details).

### SUMMARY

The MTD for every-3-week T-DM1+weekly paclitaxel was T-DM1 2 mg/kg+paclitaxel 80 mg/m².

With the addition of pertuzumab every 3 weeks, the MTD was T-DM1 2 mg/kg+paclitaxel 65 mg/m² weekly+pertuzumab 420 mg.

DLTs observed for T-DM1+paclitaxel were Grade 3 dehydration, Grade 3 AST/ALT elevation, and Grade 3 neutropenia.

DLTs observed for T-DM1+paclitaxel+pertuzumab were Grade 4 thrombocytopenia and Grade 3 neutropenia.

There were 13 partial responses (8 confirmed and 5 unconfirmed [including 4 unconfirmed due to insufficient follow-up time]) among 24 patients who received at least 1 dose of study treatment

### CONCLUSIONS

Toxicities of the T-DM l/paclitaxel combination precluded increasing the every-3-week T-DM1 dose to the 3.6 mg/kg dose used in single agent studies
The combination of T-DM1+paclitaxel had a manageable safety and tolerability profile
   Levels of thrombocytopenia and hepatic transaminase elevations were generally transient, reversible, and did not occur at a higher rate than that observed with T-DM1 alone.
   No unexpected safety signals or PK interactions were observed for T-DM1 or paclitaxel.
The combination showed encouraging clinical benefit in this heavily pretreated population.
The study is ongoing for MTD assessment of weekly T-DM1 in combination with paclitaxel and pertuzumab.

### Changed Criteria for Dose Limiting Toxicity (DLT) and Associated Dose Escalation

The study was continued with changes to the criteria for dose limiting toxicity (DLT) provided above in Section 2.1.7 (“Definition of Dose-Limiting Toxicity”). Specifically, the study was continued with a DLT being defined as any of the following:
Grade 4 neutropenia lasting ≧7 days
Febrile neutropenia
Any treatment-related toxicity prompting dose reduction of T-DM1 during the DLT observation period.
   The above criteria, being less stringent than the original DLT criteria set forth in Section 2.1.7, allowed for further dose escalation. Accordingly, T-DM1 is administered to patients at 2.4 mg/kg q3w or 2.4 mg/kg weekly, with paclitaxel also administered at 80 mg/m² weekly, with further dose escalation of T-DM1 and/or paclitaxel possible as described herein.

Although the foregoing invention has been described in some detail by way of illustration and example for purposes of clarity of understanding, the descriptions and examples should not be construed as limiting the scope of the invention. The disclosures of all patent and scientific literatures cited herein are expressly incorporated in their entirety by reference.

### REFERENCES

Abrams J S, Vena D A, Baltz J, et al. Paclitaxel activity in heavily pretreated breast cancer: a National Cancer Institute Treatment Referral Center trial. J Clin Oncol 1995; 13:2056-65.
Baselga J, Fumoleua P, Verma S, et al. A Phase II trial of trastuzumab and pertuzumab in patients with HER2-positive metastatic breast cancer that had progressed during trastuzumab therapy: full response data. European Society of Medical Oncology, Stockholm, Sweden, Sep. 12-16, 2008.
Bullock K and Blackwell K. Clinical efficacy of taxane-trastuzumab combination regimens for HER-2-positive metastatic breast cancer. Oncologist 2008; 13:515-25.
Burris H, Yardley D, Jones S, et al. Phase II trial of trastuzumab followed by weekly paclitaxel/carboplatin as first-line treatment for patients with metastatic breast cancer. J Clin Oncol 2004; 22:1621-9.
Cho H S, Mason K, Ramyar K X, et al. Structure of the extracellular region of HER2 alone and in complex with the Herceptin Fab. Nature 2003; 421:756-60.
Church D N, Modqil R, Guglani S, et al. Extended survival in women with brain metastases from HER2 overexpressing breast cancer. Am J Clin Oncol 2008; 31:250-4.
Cobleigh M A, Vogel C L, Tripathy D, et al. Multinational study of the efficacy and safety of humanized anti-HER2 monoclonal antibody in women who have HER2-overexpressing metastatic breast cancer that has progressed after chemotherapy for metastatic disease. J Clin Oncol 1999; 17:2639-48.
Cortes J, Baselga J, Wardley A, et al. Open-label, randomized, Phase II study of pertuzumab in patients with metastatic breast cancer with low expression of HER2 [abstract]. J Clin Oncol 2005; 23:3068.
Fountzilas G, Athanassiades A, Giannakakis T, et al. A phase II study of paclitaxel in advanced breast cancer resistant to anthracyclines. Eur J Cancer 1996; 32A:47-51.
Fountzilas G, Tsavdaridis D, Kalogera-Fountzila A, et al. Weekly paclitaxel as first-line chemotherapy and trastuzumab in patients with advanced breast cancer. A Hellenic Cooperative Oncology Group phase II study. Ann Oncol 2001; 12:1545-51.
Franidin M C, Carey K D, Vajdos F F, et al. Insights into ErbB signaling from the structure of the ErbB2-pertuzumab complex. Cancer Cell 2004; 5:317-28
Gasparini G, Gion M, Mariani L, et al. Randomized Phase II trial of weekly paclitaxel alone versus trastuzumab plus weekly paclitaxel as first-line therapy of patients with Her-2 positive advanced breast cancer. Breast Cancer Res Treat 2007; 101:355-65.
Gelmon K A, Fumoleau P, Verma S, et al. Results of a Phase II trial of trastuzumab and pertuzumab in patients with HER2-positive metastatic breast cancer who had progressed during trastuzumab therapy [abstract]. J Clin Oncol 2008; 26:1026.
Gori S, Colozza M, Mosconi A M, et al. Phase II study of weekly paclitaxel and trastuzumab in anthracycline- and taxa ne-p retreated patients with HER2-overexpressing metastatic breast cancer. Br J Cancer 2004; 90:36-40,
Hanahan D, Weinberg R A. The hallmarks of cancer. Cell 2000; 100:57-70.
Holmes F A, Walters R S, Theriault R L, et al. Phase II trial of taxol, an active drug in the treatment of metastatic breast cancer. J Natl Cancer Inst 1991; 83:1797-805.
Jemal A, Thun M J, Ries L A, et al. Annual report to the nation on the status of cancer, 1975-2005, featuring trends in lung cancer, tobacco use, and tobacco control. J Natl Cancer Inst 2008; 100: 1672-94.
Jones S E, Erban J, Overmoyer B, et al. Randomized phase III study of docetaxel compared with paclitaxel in metastatic breast cancer. J Clin Oncol 2005; 23:5542-51.
Kufe D W, Frei E III, Holland J F, et al., editors. Holland-Frei Cancer Medicine. 7th ed. Columbia: BC Decker Inc. 2006.
Marty M, Cognetti F, Maraninchi D, et al. Randomized phase II trial of the efficacy and safety of trastuzumab combined with docetaxel in patients with human epidermal growth factor receptor 2-positive metastatic breast cancer administered as first-line treatment: the M77001 Study Group. J Clin Oncol 2005; 23:4265-74.
Moldoveanu Z, Huang W-Q, Kulhavy R, et al. Human male genital tract secretions: both mucosal and systemic immune compartments contribute to the humoral immunity. J Immunol 2005; 175:4127-36.
Nabholtz J M, Gelmon K, Bontenbal M, et al. Multicenter, randomized comparative study of two doses of paclitaxel in patients with metastatic breast cancer. J Clin Oncol 1996; 14:1858-67.
Perez E A, Suman V J, Rowland K M, et al. Two concurrent phase II trials of paclitaxel/carboplatin/trastuzumab (weekly or every-3-week schedule) as first-line therapy in women with HER2-overexpressing metastatic breast cancer: NCCTG study 983252. Clin Breast Cancer 2005; 6:425-32.
Piccart-Gebhart M J, Procter M, Leyland-Jones B, et al. Trastuzumab after adjuvant chemotherapy in HER2-positive breast cancer. N Engl J Med 2005; 353:1659-72.
Portera C C, Walshe J M, Rosing, D R, et al. Cardiac toxicity and efficacy of trastuzumab combined with pertuzumab in patients with trastuzumabinsensitive human epidermal growth factor receptor 2-positive metastatic breast cancer. Clin Cancer Res 2008; 14: 2710-6.
Robert N, Leyland-Jones B, Asmar L, et al. Randomized phase III study of trastuzumab, paclitaxel, and carboplatin compared with trastuzumab and paclitaxel in women with HER-2-overexpressing metastatic breast cancer. J Clin Oncol 2006; 24:2786-92.
Romond E H, Perez E A, Bryant J, et al. Trastuzumab plus adjuvant chemotherapy for operable HER2-positive breast cancer. N Engl J Med 2005; 353:1673-84.
Seidman A D, Tiersten A, Hudis C, et al. Phase II trial of paclitaxel by 3-hour infusion as initial and salvage chemotherapy for metastatic breast cancer. J Clin Oncol 1995; 13:2575-81.
Seidman A D, Formier M N, Esteva F J, et al. Weekly trastuzumab and paclitaxel therapy for metastatic breast cancer with analysis of efficacy by HER2 immunophenotype and gene amplification. J Clin Oncol 2001; 19:2587-95.
Slamon D J, Clark G M, Wong S G, et al. Human breast cancer: correlation of relapse and survival with amplification of the HER-2/neu oncogene. Science 1987; 235:177-82.
Slamon D J, Leyland-Jones B, Shak S, et al. Use of chemotherapy plus a monoclonal antibody against HER2 for metastatic breast cancer that overexpresses HER2. N Engl J Med 2001; 344:783-92.
Vogel C L, Cobleigh M A, Tripathy D, et al. Efficacy and safety of trastuzumab as a single agent in first-line treatment of HER2-overexpressing metastatic breast cancer. J Clin Oncol 2002; 20:719-26.
Wilson W H, Berg S L, Bryant G, et al. Paclitaxel in doxorubicin-refractory or mitoxantrone-refractory breast cancer: a phase I/II trial of 96-hour infusion. J Clin Oncol 1994; 12:1621-9.

1. A method of treating HER2-positive cancer comprising administering a combination of paclitaxel and T-DM1 to a human patient having HER2-positive cancer, wherein paclitaxel is administered at a dosage of 65 mg/m² or 80 mg/m² weekly and T-DM1 is administered at a dosage ranging from 2.4-3.6 mg/kg every three weeks.

2. The method of claim 1, wherein T-DM1 is administered at a dosage selected from 2.4, 3.0 and 3.6 mg/kg every three weeks.

3. The method of claim 2 wherein paclitaxel is administered at a dosage of 80 mg/m² weekly and T-DM1 is administered at a dosage selected from 2.4, 3.0 and 3.6 mg/kg every three weeks.

4. The method of claim 2 wherein paclitaxel is administered at a dosage of 65 mg/m² weekly and T-DM1 is administered at a dosage selected from 2.4, 3.0 and 3.6 mg/kg every three weeks.

5. The method of claim 3 further comprising administering 420 mg pertuzumab every three weeks.

6. The method of claim 5, wherein an initial loading dose of 840 mg of pertuzumab is administered, followed by the administering of 420 mg pertuzumab every three weeks.

7. The method of claim 4 further comprising administering 420 mg pertuzumab every three weeks.

8. The method of claim 7, wherein an initial loading dose of 840 mg of pertuzumab is administered, followed by the administering of 420 mg pertuzumab every three weeks.

9. The method of claim 1, wherein the HER2-positive cancer is HER2-positive breast cancer.

10. The method of claim 9, wherein the HER2-positive breast cancer is metastatic.

11. The method of claim 1, wherein the HER2-positive cancer expresses HER2 at a 3+ level.

12. A method of treating HER2-positive cancer comprising administering a combination of paclitaxel and T-DM1 to a human patient having HER2-positive cancer, wherein paclitaxel is administered at a dosage of 65 mg/m² or 80 mg/m² weekly and T-DM1 is administered at a dosage between 1.2-2.4 mg/kg weekly.

13. The method of claim 12 wherein paclitaxel is administered at a dosage of 65 mg/m² or 80 mg/m² weekly and T-DM1 is administered at a dosage of 1.2, 1.6, 2.0 or 2.4 mg/kg weekly.

14. The method of claim 13 wherein paclitaxel is administered at a dosage of 65 mg/m² weekly and T-DM1 is administered at a dosage of 2.4 mg/kg weekly.

15. The method of claim 14 further comprising administering 420 mg pertuzumab every three weeks.

16. The method of claim 15, wherein an initial loading dose of 840 mg of pertuzumab is administered, followed by the administering of 420 mg pertuzumab every three weeks.

17. The method of claim 12, wherein the HER2-positive cancer is HER2-positive breast cancer.

18. The method of claim 17, wherein the HER2-positive breast cancer is metastatic.

19. The method of any of claim 12, wherein the HER2-positive cancer expresses HER2 at a 3+ level.

20. The method of any of claim 12, wherein paclitaxel and T-DM1 are coformulated.
